(19) United States
(12) Patent Application Publication
(10) Pub. No.: US 2025/0352771 A1
(43) Pub. Date: Nov. 20, 2025

(54) CATHETER SYSTEM FOR ONE-HANDED GUIDEWIRE AND CATHETER ADVANCEMENT

(71) Applicant: Becton, Dickinson and Company, Franklin Lakes, NJ (US)

(72) Inventors: Chad Alan Tagge, Sandy, UT (US); Daniel Blanchard, Bountiful, UT (US); Clark Ragsdale, Herriman, UT (US)

(21) Appl. No.: 19/207,081

(22) Filed: May 13, 2025

Related U.S. Application Data
(60) Provisional application No. 63/648,795, filed on May 17, 2024.

(51) Int. Cl. A61M 25/09; A61M 25/00; A61M 25/06

(52) U.S. Cl. CPC A61M 25/09025; A61M 25/0097; A61M 25/0625

(57) ABSTRACT

A catheter system may be configured such that in response to the movement from a first position to a second position, a guidewire slider may push out on a catheter hub slider. The catheter hub slider is then removed from contact with a needle hub and may slide distally. The guidewire slider and the catheter hub slider may each include a tab to facilitate one-handed advancement. Another catheter system may be configured such that in response to movement of a guidewire slider from a proximal position to a distal position, a guidewire carriage uncouples from the guidewire slider. Further advancement of the guidewire slider may not advance the guidewire. Another catheter system may be configured such that in response to movement of a guidewire slider from a proximal position, the guidewire slider is configured to engage the safety housing and advance a catheter and a guidewire together.

### RELATED APPLICATIONS

This application claims the benefit of U.S. Provisional Patent Application No. 63/648,795, which was filed on May 17, 2024, which is incorporated herein in its entirety.

### BACKGROUND

Catheters are commonly used for a variety of infusion therapies. For example, catheters may be used for infusing fluids, such as normal saline solution, various medicaments, and total parenteral nutrition, into a patient. Catheters may also be used for withdrawing blood from the patient.

A common type of catheter device includes a catheter that is over-the-needle. As its name implies, the catheter that is over-the-needle may be mounted over an introducer needle having a sharp distal tip. A catheter assembly may include a catheter hub, the catheter extending distally from the catheter hub, and the introducer needle extending through the catheter. The catheter and the introducer needle may be assembled so that the distal tip of the introducer needle extends beyond the distal tip of the catheter with the bevel of the needle facing up away from skin of the patient. The catheter and introducer needle are generally inserted into a blood vessel at a shallow angle through the skin into vasculature of the patient. After the clinician confirms the catheter is within the blood vessel, the introducer needle may be removed and the catheter may left in place for future infusion and/or blood draw.

Clinicians often use a two-handed technique to place the catheter into the blood vessel. For example, when a patient presents with difficult venous and arterial line placement, a clinician may use two hands to place the catheter. Also, clinicians commonly use two hands to utilize a guidewire to help facilitate placement of the catheter. Moreover, with the availability of ultrasound-guided technology, placement of the catheter may require one hand to hold the ultrasound device and maintain visualization of the blood vessel and the other hand to hold and place the catheter, which may not be possible. In this case, the clinician often puts down the ultrasound device, losing visual vessel placement confirmation, while the clinician advances the guidewire and/or the catheter.

The subject matter claimed herein is not limited to embodiments that solve any disadvantages or that operate only in environments such as those described above. Rather, this background is only provided to illustrate one example technology area where some implementations described herein may be practiced.

### SUMMARY

The present disclosure relates generally to vascular access devices, systems, and methods. In particular, the present disclosure relates to a catheter system configured for guidewire and catheter advancement, as well as related devices and methods.

In some embodiments, the catheter system may include a catheter assembly, which may include a catheter hub. In some embodiments, the catheter hub may include a distal end, a proximal end, and a lumen extending through the distal end of the catheter hub and the proximal end of the catheter hub. In some embodiments, the catheter system may include a catheter, which may include a distal tip and which may extend from the distal end of the catheter hub. In some embodiments, the catheter system may include a needle safety clip disposed within a safety can disposed proximal to the catheter hub.

In some embodiments, the catheter system may include a housing, which may include a distal end, a proximal end, and a slot disposed between the distal end of the housing and the proximal end of the housing. In some embodiments, the catheter system may include a needle hub fixed to the housing. In some embodiments, the catheter system may include an introducer needle, which may include a sharp distal tip and a proximal end. In some embodiments, the proximal end of the introducer needle may be secured within the needle hub.

In some embodiments, the catheter system may include a catheter hub slider, which may include a flexible arm. In some embodiments, the flexible arm may be within and part of a distally-extending arm of the catheter hub slider. In some embodiments, the catheter system may include a guidewire, which may include a first end and a second end. In some embodiments, the first end of the guidewire may be configured to advance distally through the introducer needle. In some embodiments, the catheter system may include a guidewire slider.

In some embodiments, the catheter system may be configured to move from a first position to a second position. In some embodiments, when the catheter system is in the first position, the guidewire slider may be disposed in the guidewire slider proximal position and configured to slide from a guidewire slider proximal position to a distal position to distally advance the guidewire. In some embodiments, the guidewire slider may include a tab extending through the slot and configured to move the guidewire slider between the guidewire proximal position and the distal position when the catheter system is in the first position.

In some embodiments, when the catheter system is in the first position, the catheter hub slider may be disposed in a catheter hub slider proximal position. In some embodiments, when the catheter system is in the first position, the flexible arm of the catheter hub slider may contact the needle hub to prevent the catheter hub slider from sliding from a catheter hub slider proximal position to a distally-advanced position.

In some embodiments, when the catheter system is in the second position, the guidewire slider may be disposed in the distal position such that the guidewire is advanced beyond the distal tip of the catheter. In some embodiments, when the catheter system is in the second position, the catheter hub slider may be disposed in the catheter hub slider proximal position. In some embodiments, when the catheter system is in the second position, the distally-extending arm of the catheter hub slider may be coupled to the catheter assembly to prevent distal movement and proximal movement of the catheter hub slider with respect to the catheter hub. In some embodiments, in response to the movement of the catheter system from the first position to the second position, the guidewire slider may push out on the flexible arm such that the flexible arm is removed from contact with the needle hub, and the catheter hub slider is configured to slide from a catheter hub slider proximal position to a distally-advanced position. In some embodiments, the catheter hub slider may include a tab extending through the slot and configured to move the catheter hub slider from the catheter hub slider proximal position to the distally-advanced position in response to movement of the catheter system from the first position to the second position. In some embodiments, when the catheter system is in the second position, the guidewire slider may be configured to slide back and forth between the guidewire slider proximal position and the distal position.

In some embodiments, the second end of the guidewire may be fixed within the housing. In some embodiments, the second end of the guidewire may be fixed within the needle hub. In some embodiments, the guidewire slider may include a U-shaped slot therethrough. In some embodiments, when the catheter system is in the first position, the guidewire may be disposed in the U-shaped slot in a double-throw configuration such that there is a 2:1 advancement ratio between the first end of the guidewire and the guidewire slider. In some embodiments, the U-shaped slot is oriented vertically within the housing. In some embodiments, the U-shaped slot may be asymmetric.

In some embodiments, the catheter may be configured to move from the second position to a third position by moving the tab of the catheter hub slider distally towards the tab of the guidewire slider but not contacting the tab of the guidewire slider. In some embodiments, when the catheter system is in the third position, the guidewire slider is disposed in the distal position such that the guidewire is advanced beyond the distal tip of the catheter. In some embodiments, when the catheter system is in the third position, the catheter hub slider may be disposed in the distally-advanced position, and distal advancement of the catheter hub slider advances the catheter assembly.

In some embodiments, the distally-advanced position of the catheter hub slider is a partially distally-advanced position. In some embodiments, the safety can may include a groove. In some embodiments, the housing may include an inner ramp. In some embodiments, the catheter system may be configured to move from the third position to a fourth position by moving the tab of the catheter hub slider distally towards the tab of the guidewire slider to contact the tab of the guidewire slider. In some embodiments, when the catheter system is in the fourth position, the guidewire slider is disposed in the distal position such that the guidewire is advanced beyond the distal tip of the catheter. In some embodiments, when the catheter system is in the fourth position, the catheter hub slider may be disposed in a fully distally-advanced position.

In some embodiments, in response to movement of the catheter system from the third position to the fourth position, the distally-extending arm of the catheter hub slider may contact the inner ramp on the inner surface of the housing to remove the distally-extending arm from the groove of the safety can such that the catheter hub slider is uncoupled from the catheter assembly. In some embodiments, the distally-extending arm may include a hook and an outwardly-extending protrusion. In some embodiments, in response to movement of the catheter system from the third position to the fourth position, the outwardly-extending protrusion of the catheter hub slider may contact the inner ramp on the inner surface of the housing to remove the hook from the groove of the safety can such that the catheter hub slider is uncoupled from the catheter assembly.

In some embodiments, the distally-extending arm may be a first distally-extending arm. In some embodiments, the catheter hub slider may include a second distally-extending arm opposite the first distally-extending arm. In some embodiments, in response to movement of the catheter system from the third position to the fourth position, the second distally-extending arm of the catheter hub slider may contact the inner ramp on the inner surface of the housing to remove the second distally-extending arm from the groove of the safety can such that the catheter hub slider is uncoupled from the catheter assembly.

In some embodiments, when the catheter system is in the fourth position, the housing may be moved proximally with respect to the catheter assembly to withdraw the introducer needle from the catheter assembly. In some embodiments, in response to proximal withdrawal of the introducer needle past the needle safety clip, the needle safety clip may uncouple from the catheter hub. In some embodiments, when the catheter system is in the first position, the second position, the third position, and the fourth position, the introducer needle may extend through the catheter. In some embodiments, the flexible arm may include a fin tapered inwardly in a distal direction. In some embodiments, in response to the movement of the catheter system from the first position to the second position, the guidewire slider may push out on the fin such that the flexible arm is removed from contact with the needle hub, and the catheter hub slider is configured to slide from the catheter hub slider proximal position to the distally-advanced position.

In some embodiments, the flexible arm may be a first flexible arm. In some embodiments, the catheter hub slider may include a second flexible arm opposite the first flexible arm. In some embodiments, the second flexible arm of the catheter hub slider may contact the needle hub to prevent the catheter hub slider from sliding from the catheter hub slider proximal position to the distally-advanced position. In some embodiments, in response to the movement of the catheter system from the first position to the second position, the guidewire slider may push out on the second flexible arm such that the second flexible arm is removed from contact with the needle hub, and the catheter hub slider is configured to slide from a catheter hub slider proximal position to a distally-advanced position. In some embodiments, the catheter assembly may include another safety clip disposed within the safety can and configured to trap the sharp distal tip of the introducer needle in response to proximal withdrawal of the introducer needle.

In some embodiments, a catheter system may include a catheter assembly, which may include a catheter hub. In some embodiments, the catheter hub may include a distal end, a proximal end, and a lumen extending through the distal end of the catheter hub and the proximal end of the catheter hub. In some embodiments, the catheter assembly may include a catheter, which may include a distal tip and may extend from the distal end of the catheter hub. In some embodiments, the catheter assembly may include a safety housing disposed proximal to the catheter hub. In some embodiments, the catheter assembly may include a needle safety clip disposed within the safety housing.

In some embodiments, the catheter system may include a main housing, which may include a distal end and a proximal end. In some embodiments, the catheter system may include a needle hub fixed within the main housing. In some embodiments, the catheter system may include an introducer needle, which may include a sharp distal tip and a proximal end. In some embodiments, the proximal end of the introducer needle may be secured within the needle hub.

In some embodiments, the catheter system may include a guidewire, which may include a first end and a second end. In some embodiments, the first end of the guidewire may be configured to advance distally through the introducer needle. In some embodiments, the second end of the guidewire may be fixed within the main housing. In some embodiments, the catheter system may include a guidewire slider. In some embodiments, the catheter system may include a guidewire carriage. In some embodiments, the guidewire carriage may include a U-shaped slot therethrough.

In some embodiments, the guidewire slider may be configured to move from a proximal position to a distal position to distally advance the guidewire. In some embodiments, when the guidewire slider is disposed in the distal position, the guidewire is advanced beyond the distal tip of the catheter. In some embodiments, in response to the movement of the guidewire slider from the proximal position to the distal position, the guidewire carriage may uncouple from the guidewire slider such that further advancement of the guidewire slider with respect to the main housing does not advance the guidewire. In some embodiments, when the guidewire slider is in the proximal position, the guidewire may be disposed in the U-shaped slot in a double-throw configuration such that there is a 2:1 advancement ratio between the first end of the guidewire and the guidewire slider.

In some embodiments, the guidewire carriage may include a tab that interacts with a feature on an inner surface of the guidewire slider such that the guidewire slider pushes the guidewire carriage when the guidewire slider moves distally from the proximal position. In some embodiments, in response to the movement of the guidewire slider from the proximal position to the distal position, the tab may be aligned with a recess in the housing such that the tab lowers into the recess and the guidewire carriage uncouples from the guidewire slider.

In some embodiments, when the guidewire slider is in the distal position, the guidewire slider may be configured to engage the safety housing such that further distal advancement of the guidewire slider from the distal position advances the catheter with respect to the main housing. In some embodiments, the guidewire slider may be configured to engage the safety housing between the distal position and the proximal position to advance the catheter and the guidewire at a same time. In some embodiments, in response to proximal withdrawal of the introducer needle and activation of the needle safety clip, the catheter hub may be configured to advance distally with respect to the guidewire slider and the safety housing.

In some embodiments, a catheter system may include a catheter assembly, which may include a catheter hub. In some embodiments, the catheter hub may include a distal end, a proximal end, and a lumen extending through the distal end of the catheter hub and the proximal end of the catheter hub. In some embodiments, the catheter assembly may include a catheter, which may include a distal tip and extending from the distal end of the catheter hub.

In some embodiments, the catheter system may include a safety housing disposed proximal to the catheter hub. In some embodiments, the catheter system may include a needle safety clip disposed within the safety housing. In some embodiments, the catheter system may include a main housing, which may include a distal end and a proximal end. In some embodiments, the catheter system may include a needle hub fixed within the main housing. In some embodiments, the catheter system may include an introducer needle, which may include a sharp distal tip and a proximal end. In some embodiments, the proximal end of the introducer needle may be secured within the needle hub.

In some embodiments, the catheter system may include a guidewire, which may include a first end and a second end. In some embodiments, the first end of the guidewire may be configured to advance distally through the introducer needle. In some embodiments, the second end of the guidewire may be fixed within the main housing.

In some embodiments, the catheter system may include a guidewire slider. In some embodiments, the catheter system may include a guidewire carriage, which may be permanently coupled to the guidewire slider. In some embodiments, the guidewire slider may be configured to move from a proximal position to a distal position to distally advance the guidewire. In some embodiments, when the guidewire slider is disposed in the distal position, the guidewire may be advanced beyond the distal tip of the catheter. In some embodiments, when the guidewire slider moves distal to the proximal position and before the guidewire slider reaches the distal position, the guidewire slider may be configured to engage the safety housing such that further distal advancement of the guidewire slider to the distal position advances the catheter and the guidewire at a same time.

In some embodiments, the guidewire carriage may include a U-shaped slot therethrough. In some embodiments, the guidewire may be disposed in the U-shaped slot in a double-throw configuration such that there is a 2:1 advancement ratio between the first end of the guidewire and the guidewire slider. In some embodiments, the guidewire may be single throw with respect to the guidewire carriage such that there is a 1:1 advancement ratio between the first end of the guidewire and the guidewire slider.

It is to be understood that both the foregoing general description and the following detailed description are examples and explanatory and are not restrictive of the invention, as claimed. It should be understood that the various embodiments are not limited to the arrangements and instrumentality illustrated in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE SEVERAL VIEWS OF THE DRAWINGS

Example embodiments will be described and explained with additional specificity and detail through the use of the accompanying drawings in which:

FIG. 1A is an upper perspective view of an example catheter system in an example first position, according to some embodiments;

FIG. 1B is a partial cutaway view of the catheter system in the first position, according to some embodiments;

FIG. 1C is an upper perspective view of an example catheter hub slider of the catheter system, according to some embodiments;

FIG. 1D is a lower perspective view of the catheter hub slider of the catheter system, according to some embodiments;

FIG. 1E is a cross-sectional view of the catheter system in the first position, according to some embodiments;

FIG. 1F is a side view of a portion of the catheter system in the first position, according to some embodiments;

FIG. 1G is a top, partial cutaway view of the catheter system in the first position, according to some embodiments;

FIG. 2A is an upper perspective view of an example guidewire slider, according to some embodiments;

FIG. 2B is an upper perspective view of an example guidewire, according to some embodiments;

FIG. 2C is an upper perspective view of an example needle safety clip, according to some embodiments;

FIG. 2D is an upper perspective view of another example needle safety clip, according to some embodiments;

FIG. 3A is an upper perspective view of the catheter system in an example second position, according to some embodiments;

FIG. 3B is a lower perspective view of the catheter system in the second position, according to some embodiments;

FIG. 4A is an upper perspective view of the catheter system in an example third position, according to some embodiments;

FIG. 4B is an upper perspective view illustrating an example safety can coupled to the catheter hub slider in the third position, according to some embodiments;

FIG. 5A is an upper perspective view illustrating the safety can being uncoupled from the catheter hub slider as moved to an example fourth position, according to some embodiments;

FIG. 5B is an upper perspective view of the catheter system in the fourth position, according to some embodiments;

FIG. 6A is a lower perspective view of a portion of the catheter system in the fourth position, according to some embodiments;

FIG. 6B is an upper perspective view of the catheter system having an example introducer needle proximally withdrawn past the needle safety clip such that the needle safety clip is uncoupled from the catheter hub;

FIG. 7A is an upper perspective view of the catheter system having an example slot, the catheter system illustrated in the first position, according to some embodiments;

FIG. 7B is a cross-sectional view of the catheter system having the slot, the catheter system illustrated in the first position, according to some embodiments;

FIG. 7C is an upper perspective view of the catheter system having the slot, the catheter system illustrated in the second position, according to some embodiments;

FIG. 7D is an upper perspective view of the catheter system having the slot, the catheter system illustrated in the third position, according to some embodiments; and

FIG. 7E is an upper perspective view of the catheter system having the slot, the catheter system illustrated in the fourth position, according to some embodiments;

FIG. 8A is an upper perspective view of the catheter system, illustrating an example tab of the guidewire slider and an example tab of the catheter hub slider when the catheter system is in the first position, according to some embodiments;

FIG. 8B is a cross-sectional view of the catheter system, illustrating the tab of the guidewire slider and the tab of the catheter hub slider when the catheter system is in the first position, according to some embodiments;

FIG. 8C is another cross-sectional view of the catheter system, illustrating the tab of the guidewire slider and the tab of the catheter hub slide when the catheter system is in the first position, according to some embodiments;

FIG. 8D is an upper perspective view of the catheter system, illustrating the tab of the guidewire slider and the tab of the catheter hub slider when the catheter system is in the second position, according to some embodiments;

FIG. 8E is an upper perspective view of the catheter system, illustrating the tab of the guidewire slider and the tab of the catheter hub slider when the catheter system is in the third position, according to some embodiments;

FIG. 8F is an upper perspective view of the catheter system, illustrating the tab of the guidewire slider and the tab of the catheter hub slider when the catheter system is in the fourth position, according to some embodiments;

FIG. 9A is an upper perspective view of another example catheter system, illustrating an example guidewire slider in an example proximal position, according to some embodiments;

FIG. 9B is a cross-sectional view of the catheter system of FIG. 9A, illustrating the guidewire slider in the proximal position, according to some embodiments;

FIG. 10A is an upper perspective view of another example catheter system, illustrating an example guidewire slider in an example proximal position, according to some embodiments;

FIG. 10B is an upper perspective view of the catheter system of FIG. 10A, illustrating the guidewire slider in an example distal position, according to some embodiments;

FIG. 10C is a cross-sectional view of the catheter system of FIG. 10A, illustrating the guidewire slider in an example distal position, according to some embodiments;

FIG. 11A is an upper perspective view of an example catheter system, illustrating an example guidewire slider in an example proximal position, according to some embodiments;

FIG. 11B is an upper perspective view of the catheter system of FIG. 11A, illustrating the guidewire slider in a position between the proximal position and an example distal position, according to some embodiments;

FIG. 11C is an upper perspective view of the catheter system of FIG. 11A, illustrating the guidewire slider in the distal position, according to some embodiments;

FIG. 11D is an upper perspective view of the catheter system of FIG. 11A, illustrating the guidewire slider further distally advanced from the distal position such that the catheter is distally advanced, according to some embodiments;

FIG. 11E is an upper perspective view of the catheter system of FIG. 11A, illustrating the guidewire slider even further distally advanced from the FIG. 11D such that the catheter is further distally advanced, according to some embodiments;

FIG. 11F is an upper perspective view of the catheter system of FIG. 11A, illustrating the introducer needle retracted, according to some embodiments;

FIG. 12A is a cross-sectional view of the catheter system of FIG. 11A, illustrating the guidewire slider in the proximal position with an example guidewire and example introducer needle removed for illustrative purposes, according to some embodiments;

FIG. 12B is a cross-sectional view of the catheter system of FIG. 11A, illustrating the guidewire slider in the proximal position, according to some embodiments;

FIG. 13A is an exploded view of the catheter system of FIG. 11A, according to some embodiments;

FIG. 13B is a lower perspective view of the guidewire slider, according to some embodiments;

FIG. 13C is an upper perspective view of an example portion of a guidewire carriage, according to some embodiments;

FIG. 13D is a side view of the portion of the guidewire carriage, according to some embodiments;

FIG. 13E is a top view of an example bottom portion of a main housing, according to some embodiments;

FIG. 13F is a lower perspective view of an example top portion of the main housing, according to some embodiments; and

FIG. 14 is an upper perspective view of a clinician inserting the catheter system of 11A into a patient and advancing the guidewire and the catheter with a single hand, according to some embodiments.

### DESCRIPTION OF EMBODIMENTS

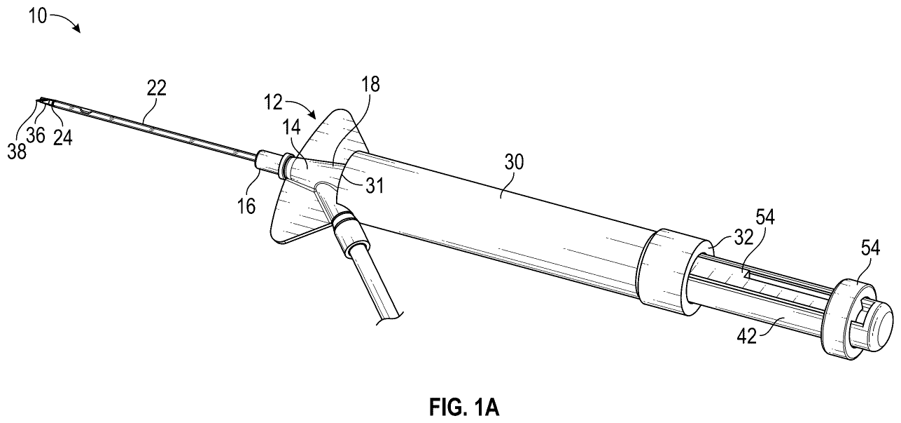

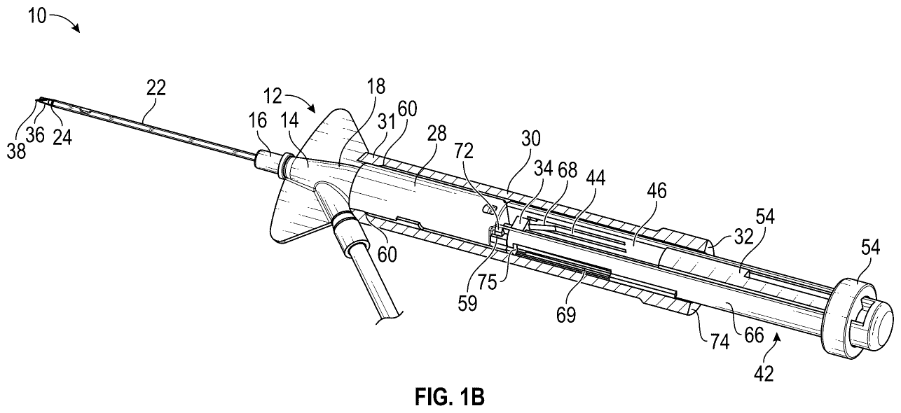

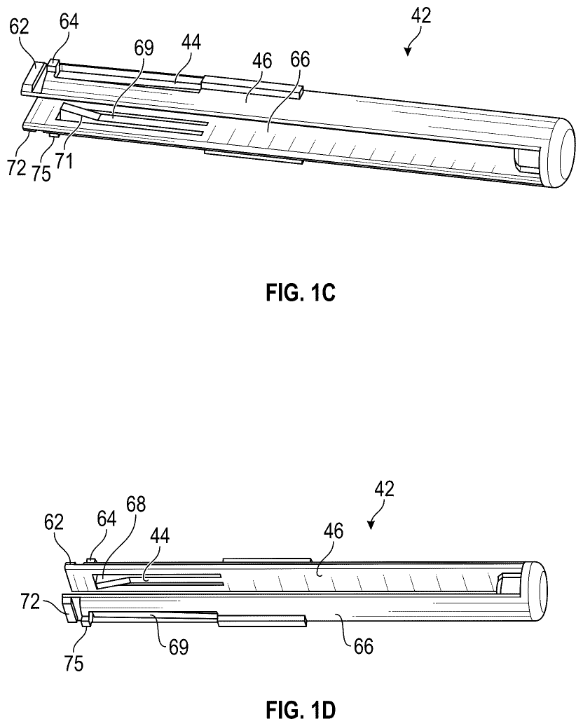

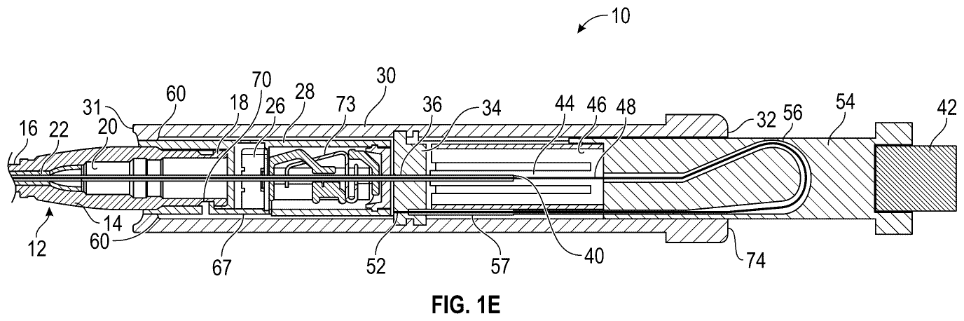

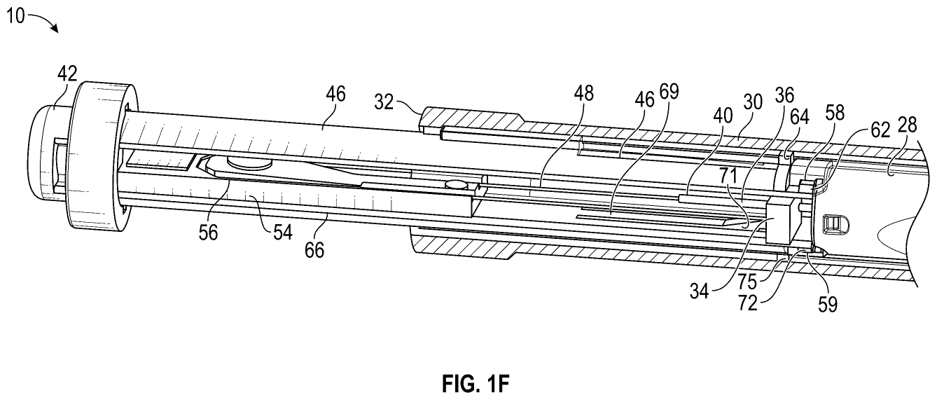

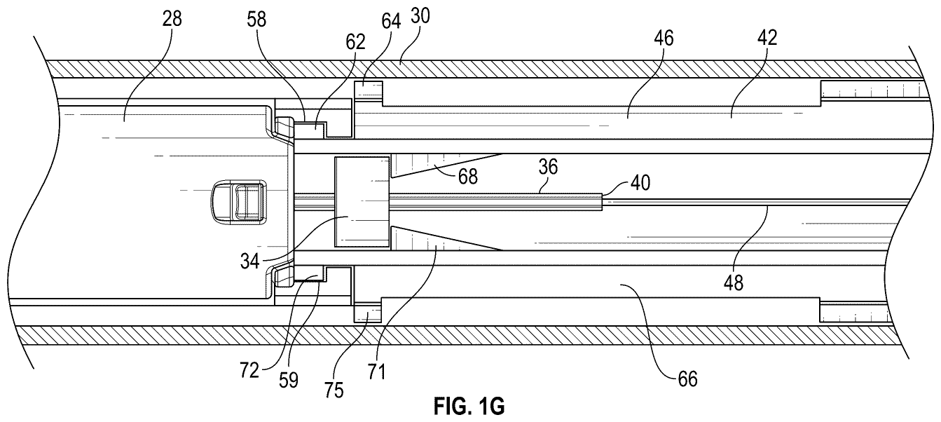

Referring now to FIGS. 1A-6B, in some embodiments, a catheter system 10 may include a catheter assembly 12, which may include a catheter hub 14. In some embodiments, the catheter hub 14 may include a distal end 16, a proximal end 18, and a lumen 20 extending through the distal end 16 of the catheter hub 14 and the proximal end 18 of the catheter hub 14. In some embodiments, the catheter system 10 may include a catheter 22, which may include a distal tip 24 and which may extend from the distal end 16 of the catheter hub 14. In some embodiments, the catheter system 10 may include a needle safety clip 26 disposed within a safety can 28 disposed proximal and/or proximate to the catheter hub 14.

In some embodiments, the catheter system 10 may be integrated, having an extension tube integrated with a side port, such as, for example, BD NEXIVA™ Closed IV Catheter System, available from Becton Dickinson and Company of Franklin Lakes, New Jersey. In other embodiments, the catheter system 10 may be non-integrated, such as, for example, the BD INSYTE™ AUTOGUARD™ Shielded IV Catheter, available from Becton Dickinson and Company of Franklin Lakes, New Jersey.

In some embodiments, the catheter system 10 may include a housing 30, which may include a distal end 31 and a proximal end 32. In some embodiments, the catheter system 10 may include a needle hub 34 fixed to the housing 30. In some embodiments, the catheter system 10 may include an introducer needle 36, which may include a sharp distal tip 38 and a proximal end 40. In some embodiments, the proximal end 40 of the introducer needle 36 may be secured within the needle hub 34.

In some embodiments, the catheter system 10 may include a catheter hub slider 42, which may include a flexible arm 44. In some embodiments, the flexible arm 44 may be within and part of a distally-extending arm 46 of the catheter hub slider 42. In some embodiments, the catheter system 10 may include a guidewire 48, which may include a first end 50 and a second end 52. In some embodiments, the first end 50 of the guidewire 48 may be configured to advance distally through the introducer needle 36. In some embodiments, the catheter system 10 may include a guidewire slider 54.

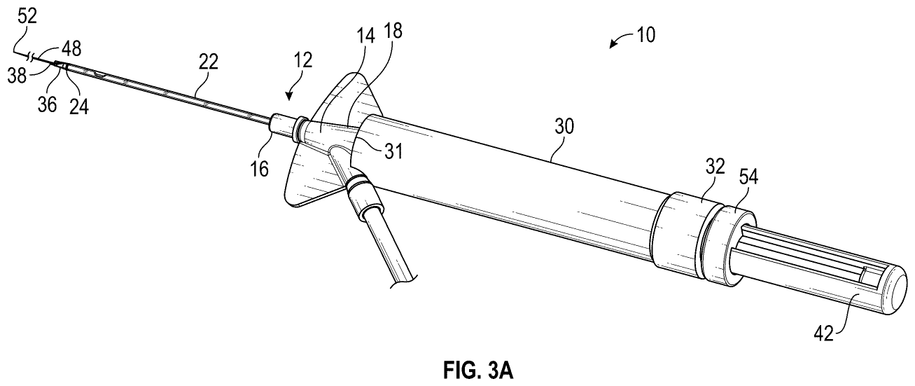

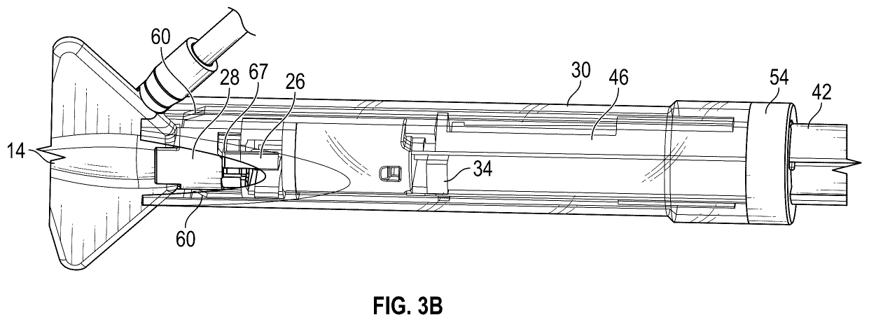

In some embodiments, the catheter system 10 may be configured to move from a first position, illustrated for example in FIGS. 1A-1B and 1E-1F, to a second position, illustrated for example in FIGS. 3A-3B. In some embodiments, when the catheter system 10 is in the first position, the guidewire slider 54 may be disposed in the guidewire slider proximal position and configured to slide from a guidewire slider proximal position to a distal position to distally advance the guidewire 48. In some embodiments, when the catheter system 10 is in the first position, the catheter hub slider 42 may be disposed in a catheter hub slider proximal position. In some embodiments, when the catheter system 10 is in the first position, the flexible arm 44 of the catheter hub slider 42 may contact the needle hub 34 to prevent the catheter hub slider 42 from sliding from a catheter hub slider proximal position to a distally-advanced position.

In some embodiments, when the catheter system 10 is in the second position, the guidewire slider 54 may be disposed in the distal position such that the guidewire 48 is advanced beyond the distal tip 24 of the catheter 22. In some embodiments, when the catheter system 10 is in the second position, the catheter hub slider 42 may be disposed in the catheter hub slider proximal position. In some embodiments, when the catheter system 10 is in the second position, the distally-extending arm 46 of the catheter hub slider 42 may be coupled to the catheter assembly 12 via the safety can 28 to prevent distal movement and proximal movement of the catheter hub slider 42 with respect to the catheter hub 14. In some embodiments, in response to the movement of the catheter system 10 from the first position to the second position, the guidewire slider 54 may push out on the flexible arm 44 such that the flexible arm 44 is removed from contact with the needle hub 34, and the catheter hub slider 42 is configured to slide from the catheter hub slider proximal position to the distally-advanced position. In some embodiments, when the catheter system 10 is in the second position, the guidewire slider 54 may be configured to slide back and forth between the guidewire slider proximal position and the distal position.

In some embodiments, the second end 52 of the guidewire 48 may be fixed within the housing 30. In some embodiments, the second end 52 of the guidewire 48 may be fixed to the housing 30. In some embodiments, the second end 52 of the guidewire 48 may be fixed within the needle hub 34. In some embodiments, the guidewire slider 54 may include a U-shaped slot 56 therethrough. In some embodiments, when the catheter system 10 is in the first position, the guidewire 48 may be disposed in the U-shaped slot 56 in a double-throw configuration such that there is a 2:1 advancement ratio between the first end 50 of the guidewire 48 and the guidewire slider 54. In some embodiments, the U-shaped slot 56 is oriented vertically within the housing 30. In some embodiments, the catheter system 10 may include a support tubing 57, which may extend from the needle hub 34 and may facilitate support of the guidewire 48.

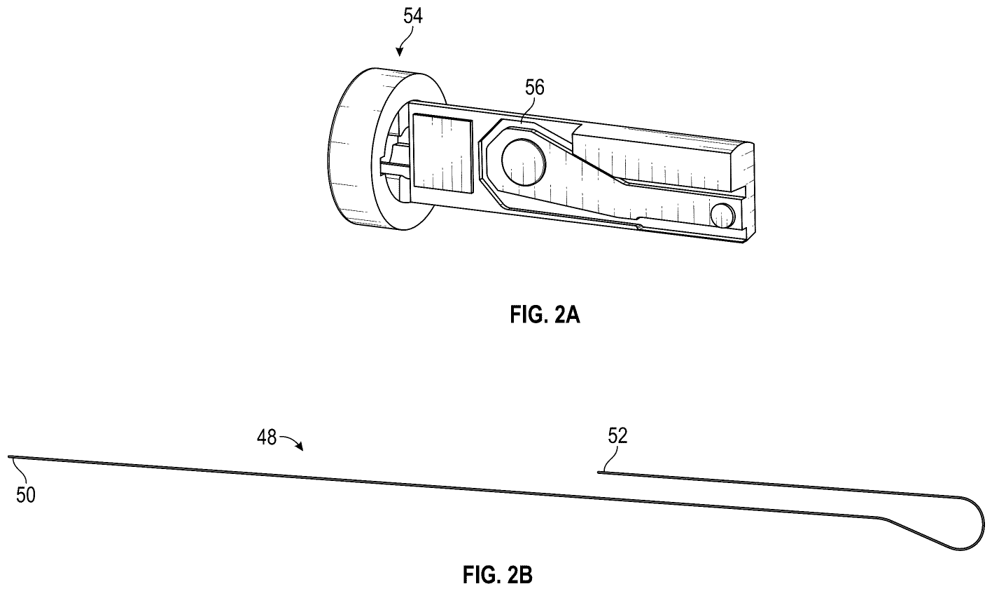

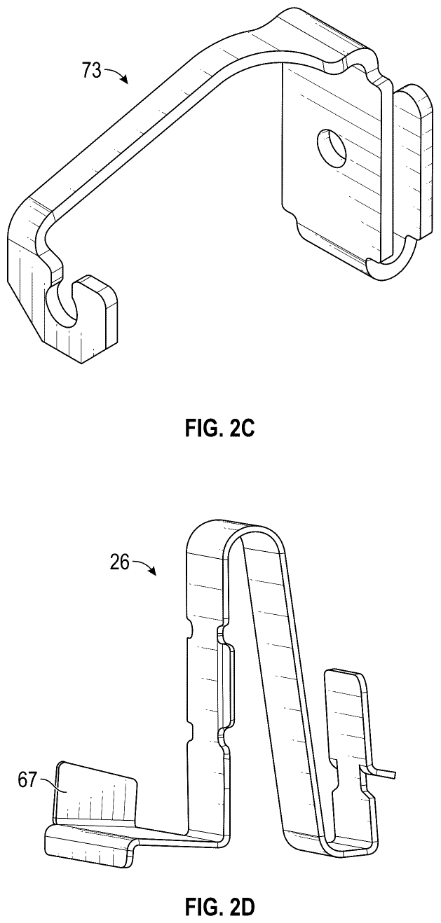

In some embodiments, the U-shaped slot 56 may be asymmetric. For example, as illustrated or example in FIG. 2A, the U-shaped slot 56 may be elongated towards a top of the catheter system 10, which may decrease an overall diameter or size of the housing 30 for gripping by a clinician.

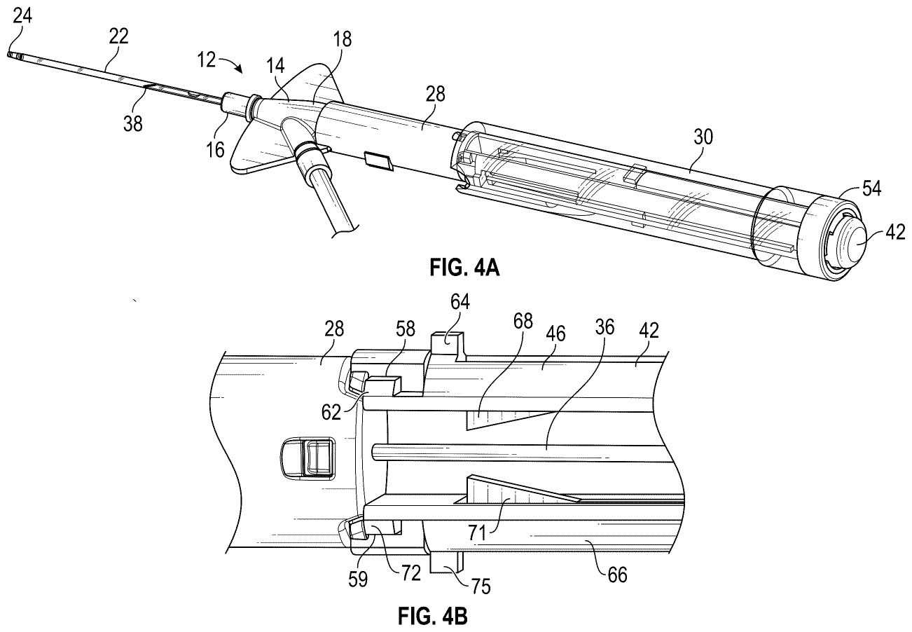

In some embodiments, the catheter 22 may be configured to move from the second position to a third position, illustrated for example in FIGS. 4A-4B. In some embodiments, when the catheter system 10 is in the third position, the guidewire slider 54 may be disposed in the distal position such that the guidewire 48 is advanced beyond the distal tip 24 of the catheter 22. In some embodiments, when the catheter system 10 is in the third position, the catheter hub slider 42 may be disposed in the distally-advanced position, and distal advancement of the catheter hub slider 42 advances the catheter assembly 12.

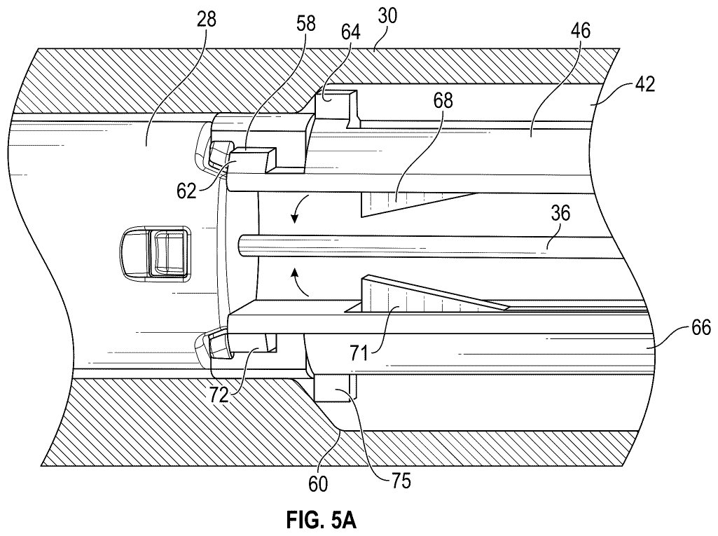

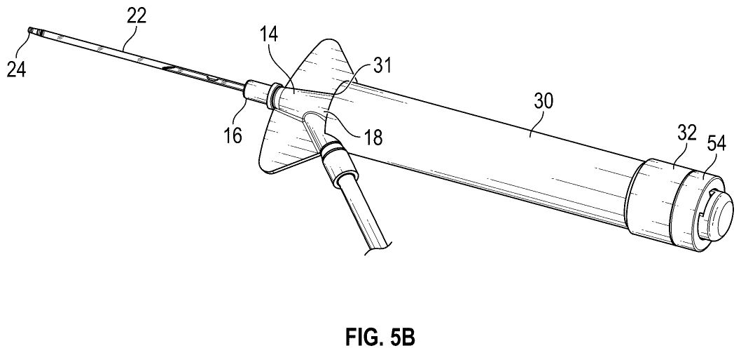

In some embodiments, the distally-advanced position of the catheter hub slider 42 is a partially distally-advanced position. In some embodiments, the safety can 28 may include one or more grooves 58. In some embodiments, the housing 30 may include an inner ramp 60. In some embodiments, the catheter system 10 may be configured to move from the third position to a fourth position, illustrated for example in FIG. 5. In some embodiments, when the catheter system 10 is in the fourth position, the guidewire slider 54 may be disposed in the distal position such that the guidewire 48 is advanced beyond the distal tip 24 of the catheter 22. In some embodiments, when the catheter system 10 is in the fourth position, the catheter hub slider 42 may be disposed in a fully distally-advanced position.

In some embodiments, in response to movement of the catheter system 10 from the third position to the fourth position, the distally-extending arm 46 of the catheter hub slider 42 may contact the inner ramp 60 on the inner surface of the housing 30 to remove the distally-extending arm 46 from the groove 58 of the safety can 28 such that the catheter hub slider 42 is uncoupled from the catheter assembly 12. In some embodiments, the distally-extending arm 46 may include a hook 62 and an outwardly-extending protrusion 64. In some embodiments, in response to movement of the catheter system 10 from the third position to the fourth position, the outwardly-extending protrusion 64 of the catheter hub slider 42 may contact the inner ramp 60 on the inner surface of the housing 30 to remove the hook 62 from the groove of the safety can such that the catheter hub slider 42 is uncoupled from the catheter assembly 12.

In some embodiments, the distally-extending arm 46 may be a first distally-extending arm. In some embodiments, the catheter hub slider 42 may include a second distally-extending arm 66 opposite the first distally-extending arm. In some embodiments, in response to movement of the catheter system 10 from the third position to the fourth position, the second distally-extending arm 66 of the catheter hub slider 42 may contact the inner ramp 60 on the inner surface of the housing 30 to remove the second distally-extending arm 66 from the groove 58 or another groove 59 of the safety can 28 such that the catheter hub slider 42 is uncoupled from the catheter assembly 12.

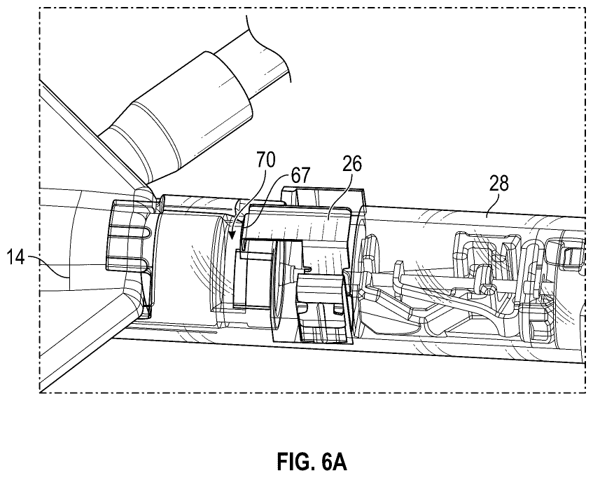

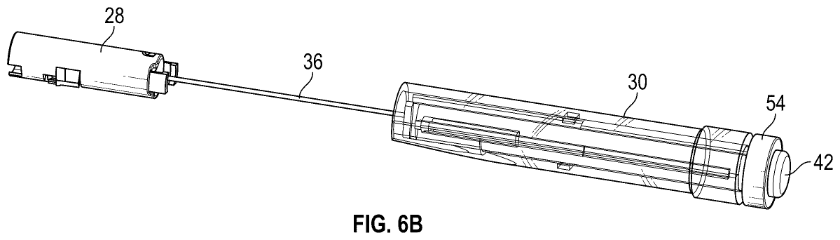

In some embodiments, when the catheter system 10 is in the fourth position, the housing 30 may be moved proximally with respect to the catheter assembly 12 to withdraw the introducer needle 36 from the catheter assembly 12. In some embodiments, in response to proximal withdrawal of the introducer needle 36 past the needle safety clip 26, the needle safety clip 26 may uncouple from the catheter hub 14. As illustrated for example in FIG. 6A, in some embodiments, the needle safety clip 26 may include a V-shaped clip with a pawl 67 inserted into a groove 70 of the catheter hub 14. In some embodiments, in response to proximal withdrawal of the introducer needle 36 past the needle safety clip 26, the V-shaped clip may expand without compression by the introducer needle 36, causing release of the pawl 67 from the groove 70 and uncoupling from the catheter hub 14.

In some embodiments, when the catheter system 10 is in the first position, the second position, the third position, and the fourth position, the introducer needle 36 may extend through the catheter 22. As illustrated for example in FIG. 1B, in some embodiments, the flexible arm 44 may include a fin 68 tapered inwardly in a distal direction. As illustrated in FIG. 5A, in some embodiments, in response to the movement of the catheter system 10 from the first position to the second position, the guidewire slider may push out on the fin 68 such that the flexible arm 44 is removed from contact with the needle hub 34, and the catheter hub slider 42 is configured to slide from the catheter hub slider proximal position to the distally-advanced position.

In some embodiments, the flexible arm 44 may be a first flexible arm and/or the fin 68 may be a first fin. In some embodiments, the catheter hub slider 42 may include a second flexible arm 69 opposite the first flexible arm, as illustrated for example in FIGS. 1C-1D. In some embodiments, the second flexible arm 69 of the catheter hub slider 42, which may include a second fin 71, may contact the needle hub 34 to prevent the catheter hub slider 42 from sliding from the catheter hub slider proximal position to the distally-advanced position, as illustrated for example in FIG. 4B. In some embodiments, the hook 62 may be a first hook, and the outwardly-extending protrusion 64 may include a first outwardly-extending protrusion. In some embodiments, the second flexible arm 69 may include a second hook and/or a second outwardly-extending protrusion.

In some embodiments, in response to the movement of the catheter system 10 from the first position to the second position, the guidewire slider 54 may push out on the second flexible arm 69 such that the second flexible arm 69 is removed from contact with the needle hub 34 similar to the first flexible arm, and the catheter hub slider 42 is configured to slide from the catheter hub slider proximal position to the distally-advanced position. In some embodiments, the catheter system 10 may include another safety clip 73 disposed within the safety can 28 and configured to trap the sharp distal tip 38 of the introducer needle 36 in response to proximal withdrawal of the introducer needle 36.

In some embodiments, the proximal end 32 of the housing 30 may include an opening 74. In some embodiments, the catheter hub slider 42 may be configured to slide within the opening 74 from the proximal catheter hub slider position to the distally-advanced position. In some embodiments, the guidewire slider 54 may be configured to slide within the opening 74 from the guidewire slider proximal position to the distal position. In some embodiments, the guidewire slider 54 may wrap around the catheter hub slider 42. In some embodiments, the guidewire slider 54 may wrap completely around the catheter hub slider 42. In some embodiments, a proximal end of the catheter hub slider 42 may include a vent plug configured to vent air, which may facilitate depression or distal movement of the catheter hub slider 42.

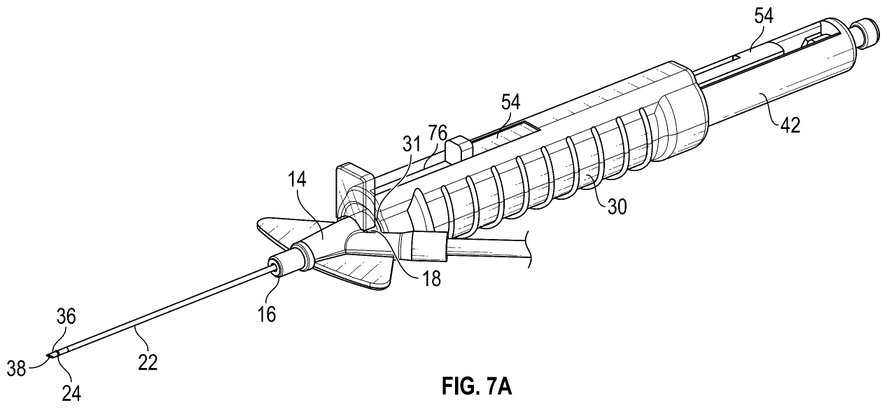

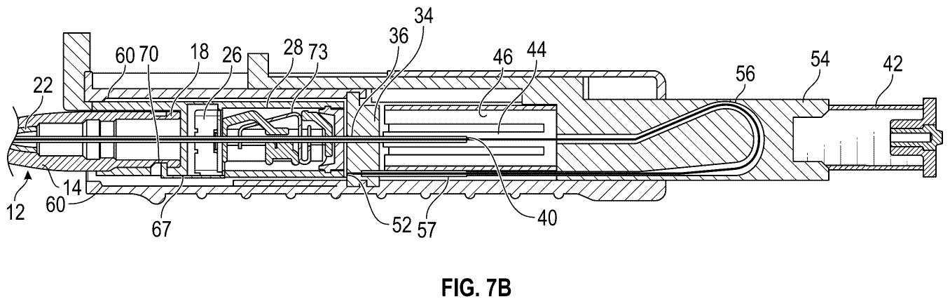

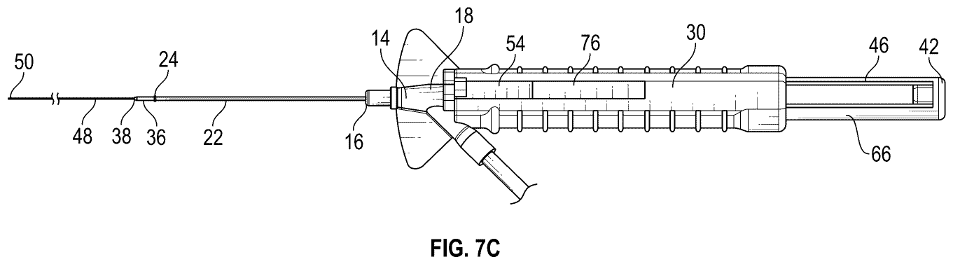

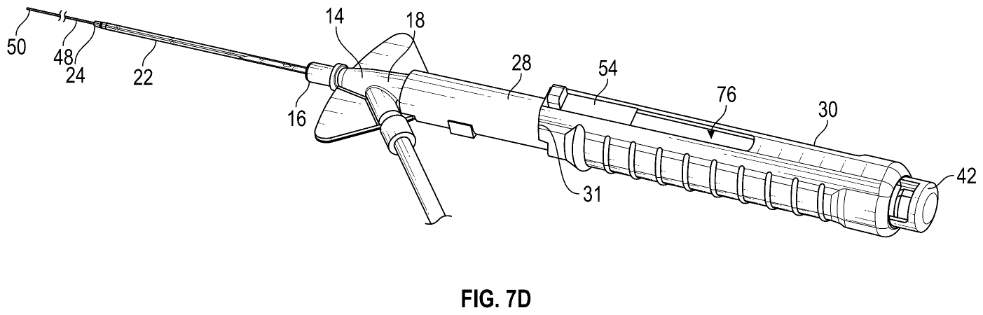

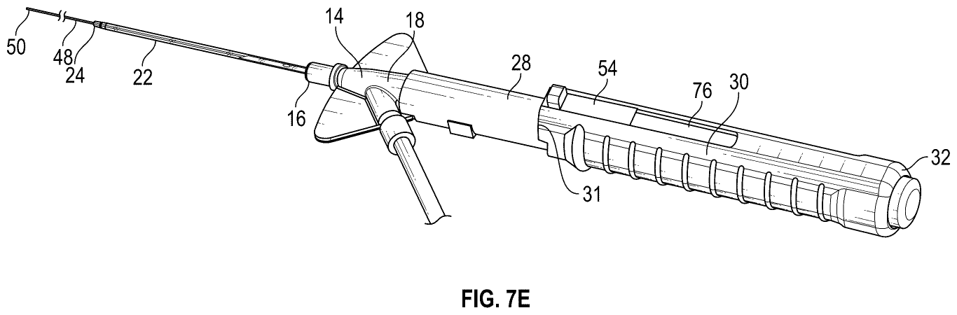

Referring now to FIGS. 7A-7E, the catheter system 10 is illustrated according to some embodiments. In some embodiments, the housing 30 may include a slot 76 disposed between the distal end 31 of the housing 30 and the proximal end 18 of the housing 30. In some embodiments, when the catheter system 10 is in the first position, the guidewire slider 54 may extend through the slot 76 and may be configured to slide along the slot 76 from the guidewire slider proximal position to the distal position to distally advance the guidewire 48. In some embodiments, when the catheter system is in the second position, the guidewire slider 54 may extend through the slot 76 and may be configured to slide along the slot 76 from the guidewire slider distal position to the catheter hub proximal position. In some embodiments, an upper surface of the guidewire slider 54 may include an upwardly-extending protrusion 78, which may facilitate advancement and/or retraction of the guidewire slider 54 by a finger of the clinician. In some embodiments, the proximal end of the catheter hub slider 42 may include the vent plug configured to vent air, which may facilitate depression or distal movement of the catheter hub slider 42.

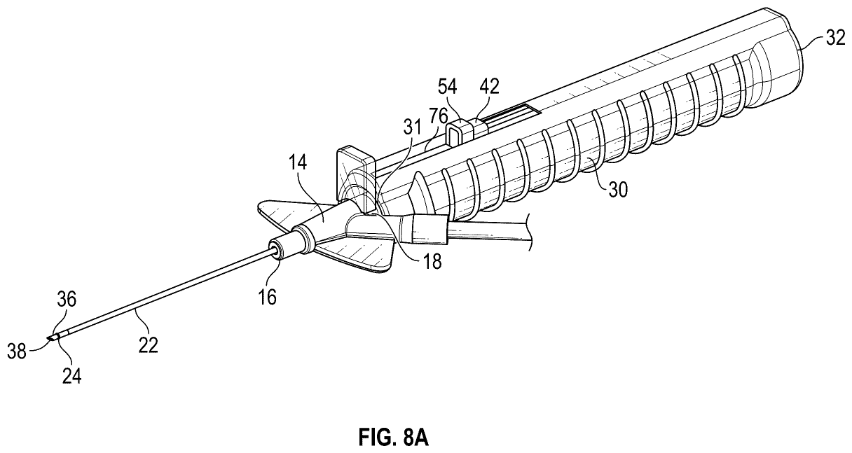

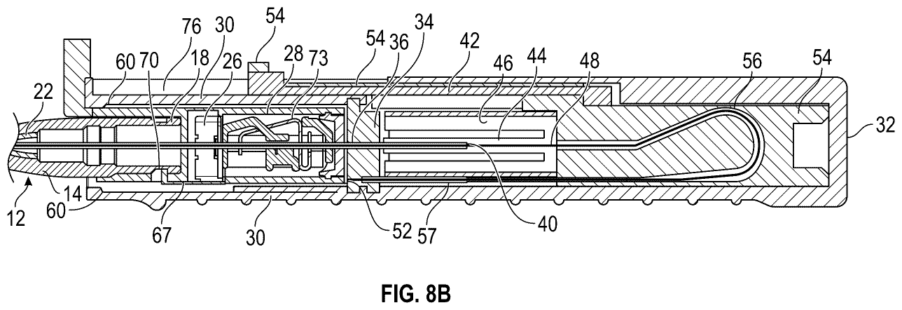

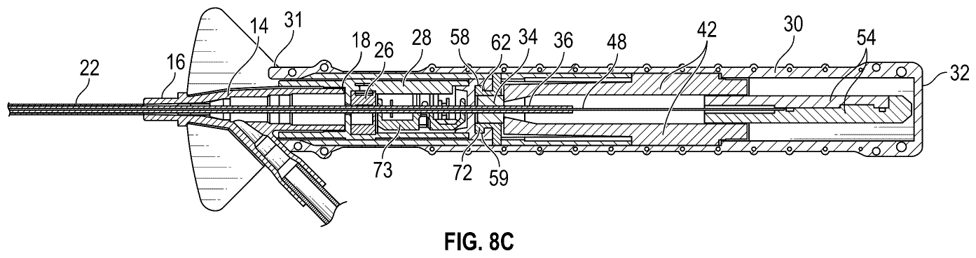

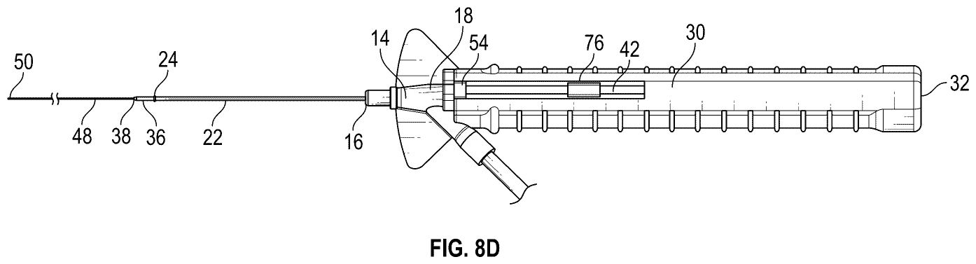

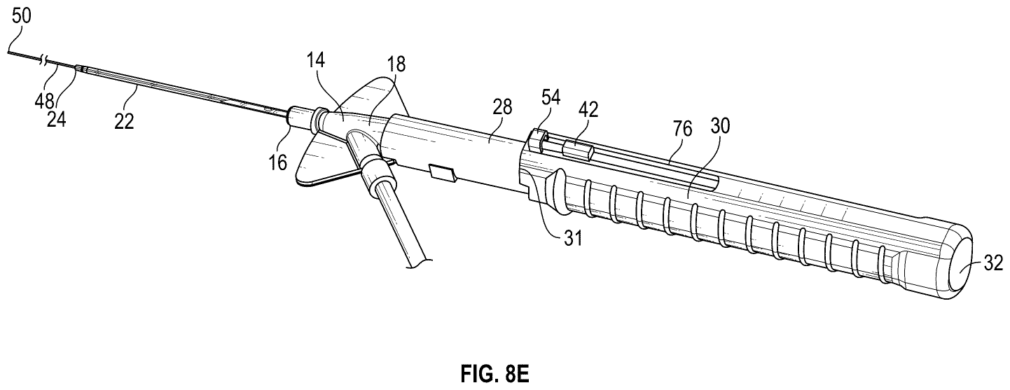

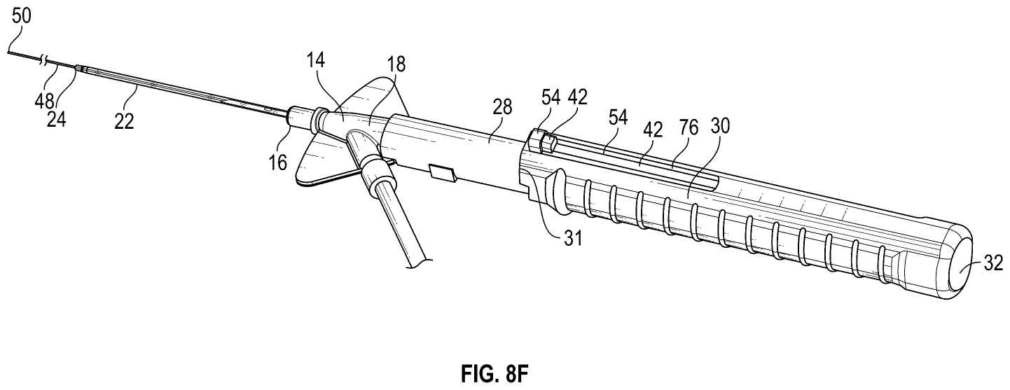

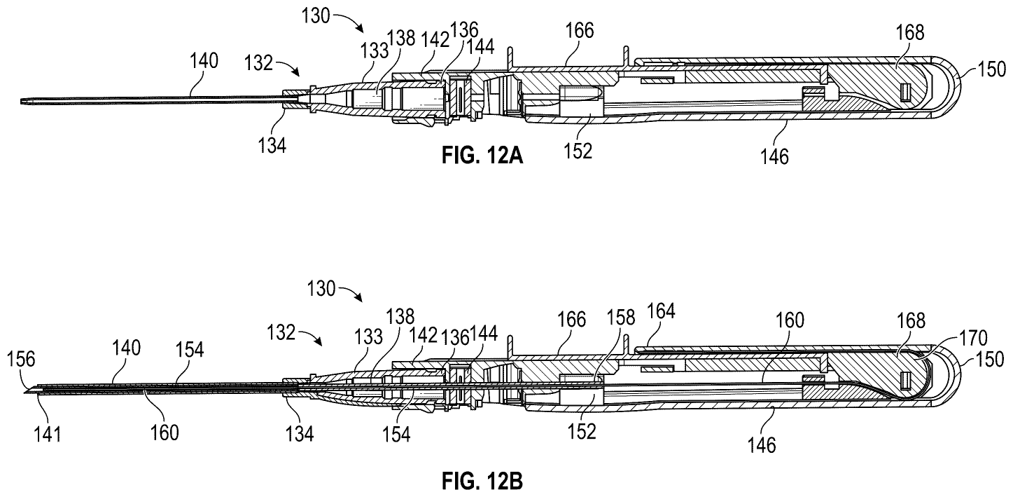

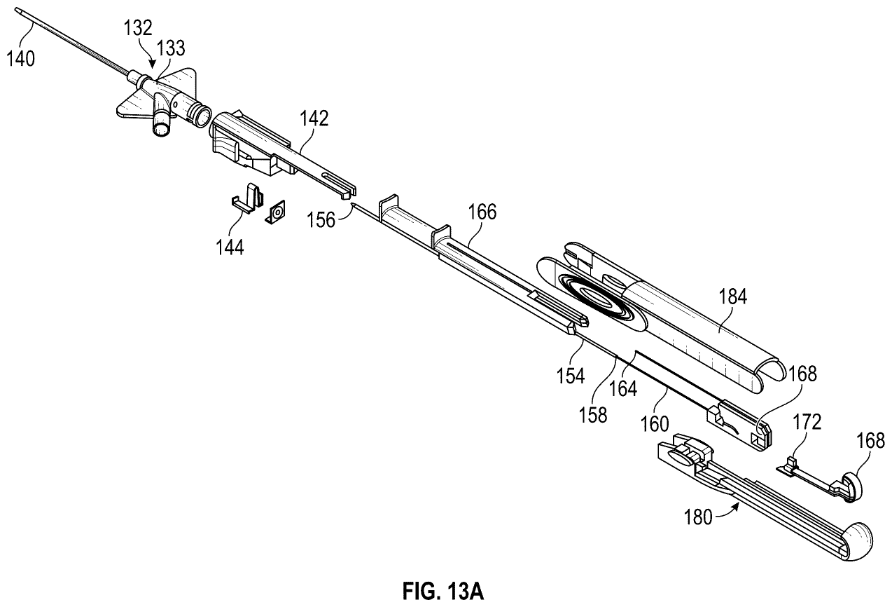

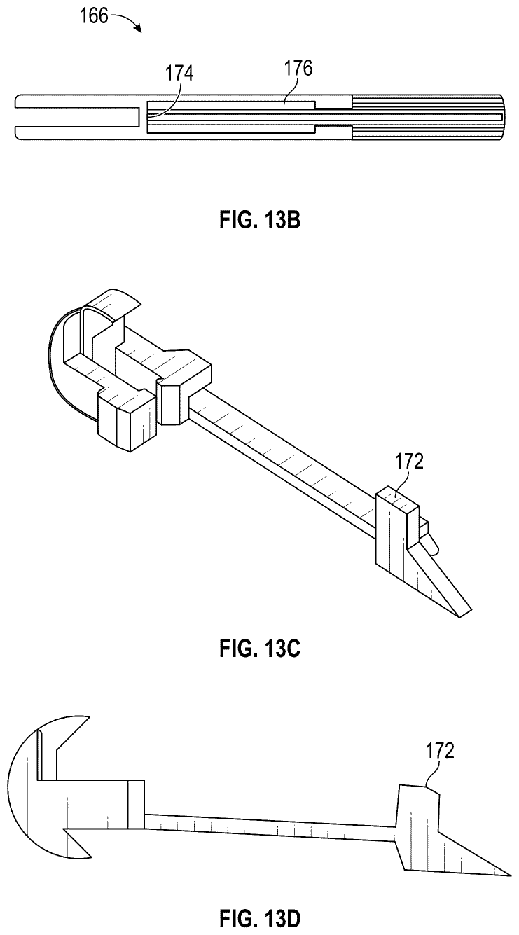

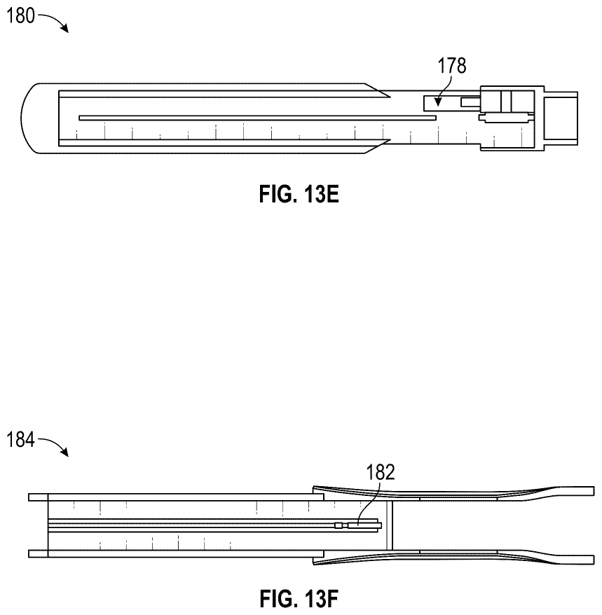

The catheter systems discussed with respect to FIGS. 8-14 may be configured to allow a clinician to place a catheter and insert a guidewire using a single hand, according to some embodiments. The catheter systems of FIGS. 8-14 may include closed catheter systems having a guidewire, a safety housing, a guidewire slider, and a chamber to visualize blood vessel access confirmation, according to some embodiments. In some embodiments, the catheter systems of FIGS. 8-14 may include significant improvements to existing venous and arterial catheter placement by allowing the clinician to use a single-handed technique to access a blood vessel, visualize blood vessel confirmation, advance the guidewire, and advance a catheter into the blood vessel, while allowing the clinician to use a second hand to hold an ultrasound probe and continuously maintain blood vessel confirmation. In some embodiments, the catheter systems of FIGS. 8-14 may facilitate the clinician having continuous blood vessel visualization via ultrasound so that the clinician does not have to blindly (without ultrasound) advance the guidewire.

In some embodiments, the catheter systems of FIGS. 8-14 may include an integrated guidewire for arterial or venous placement. In some embodiments, the catheter systems of FIGS. 8-14 may include a guidewire slider to advance the guidewire with either a single or double throw, and a hand holding a particular catheter system can advance the guidewire using the guidewire slider without needing to change grip. In some embodiments, the guidewire slider may be dual purpose; after the guidewire slider is used to advance the guidewire, the guidewire slider may again be used to advance the catheter off an introducer needle and into the blood vessel. In some embodiments, the catheter systems of FIGS. 8-14 may include passive needle safety to protect the clinician from needle sticks and/or blood exposure. In some embodiments, the catheter systems of FIGS. 8-14 may use magnetic guidance technology to facilitate introducer needle and catheter insertion, such as, for example, the CUE™ Needle Tracking System available from Becton Dickinson and Company of Franklin Lakes, New Jersey.

In some embodiments, the catheter systems of FIGS. 8-14 may include flashback tubing that may be bonded to a distal end and/or a proximal end of a housing, vented to the atmosphere, and in fluid communication with the introducer needle. In some embodiments, a portion of a flashback chamber may include a transparent wall, which may include curvature for magnification, to allow the clinician to see pulsation pressure of arterial blood to confirm successful placement in an artery and continued access to the artery. In some embodiments, the flashback chamber may be vented at any position based on how far blood flashback is to move into the flashback chamber.

Referring now to FIGS. 8A-8F, the catheter system 10 is illustrated according to some embodiments. In some embodiments, the catheter system 10 may be used for arterial or venous access. In some embodiments, the housing 30 may include the slot 76 disposed between the distal end 31 of the housing 30 and the proximal end 18 of the housing 30. In some embodiments, the catheter system 10 may be similar or identical to the catheter system 10 of one or more of FIGS. 1A-7D in terms of one or more features and/or operation. For example, the first position, the second position, the third position, and the fourth position referred to with respect to FIGS. 8A-8F may correspond to the first position, the second position, the third position, and the fourth position discussed with respect to FIGS. 1A-7D.

In some embodiments, the guidewire slider 54 and the catheter hub slider 42 may extend through the slot 76. In some embodiments, an upper surface of the guidewire slider 54 may include an upwardly-extending protrusion or tab, which may extend through the slot 76. In some embodiments, the tab may facilitate advancement and/or retraction of the guidewire slider 54 by a finger of the clinician. In some embodiments, an upper surface of the catheter hub slider 42 may include an upwardly-extending protrusion or tab, which may extend through the slot 76. In some embodiments, the tab of the catheter hub slider 42 may facilitate advancement and/or retraction of the catheter hub slider 42 by the finger of the clinician.

In some embodiments, when the catheter system 10 is in the first position (as illustrated for example, in FIGS. 8A-8C), the tab of the guidewire slider 54 may be slid along the slot 76 to move the guidewire slider 54 from the guidewire slider proximal position to the distal position to distally advance the guidewire 48. In some embodiments, when the catheter system 10 is in the second position, the tab of the guidewire slider 54 may be slid along the slot 76 from the guidewire slider distal position to or proximate the catheter hub proximal position, as illustrated, for example, in FIG. 8D.

In some embodiments, the catheter system 10 may be configured to move from the second position to the third position by moving the tab of the catheter hub slider 42 distally towards the tab of the guidewire slider 54 but not contacting the tab of the guidewire slider 54, as illustrated, for example, in FIG. 8E. In some embodiments, the catheter system 10 may be configured to move from the third position to the fourth position by moving the tab of the catheter hub slider 42 distally towards the tab of the guidewire slider 54 to contact the tab of the guidewire slider 54, as illustrated, for example, in FIG. 8F. In some embodiments, the tab of the catheter hub slider 42 may be configured to fit inside the tab of the guidewire slider 54 when the catheter system 10 is in the first position and/or the fourth position. In some embodiments, the tab of the guidewire slider 54 and the tab of the catheter hub slider 42 may move along a same slot and/or be actuated from a same location to facilitate one-handed insertion of the catheter system 10.

In some embodiments, in response to movement of the catheter system 10 from the third position to the fourth position, the distally-extending arm 46 of the catheter hub slider 42 may contact the inner ramp on the inner surface of the housing 30 to remove the distally-extending arm 46 from the groove 58 of the safety can 28 such that the catheter hub slider 42 is uncoupled from the catheter assembly 12. Thus, in FIG. 8F, the catheter hub slider 42 may be uncoupled from the catheter assembly 12 such that the catheter assembly 12 can be further advanced into the blood vessel if desired.

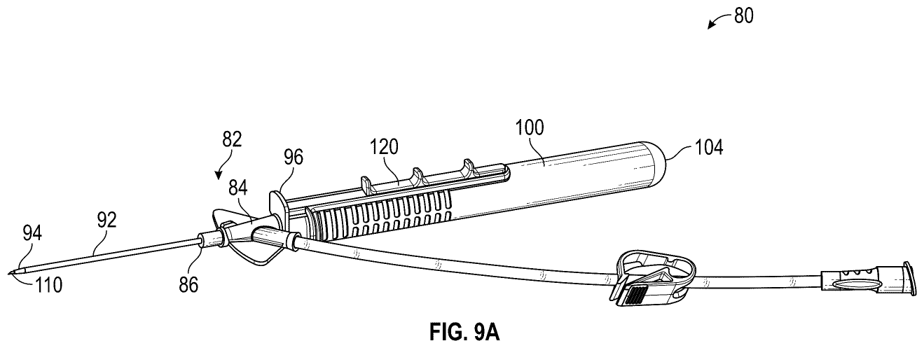

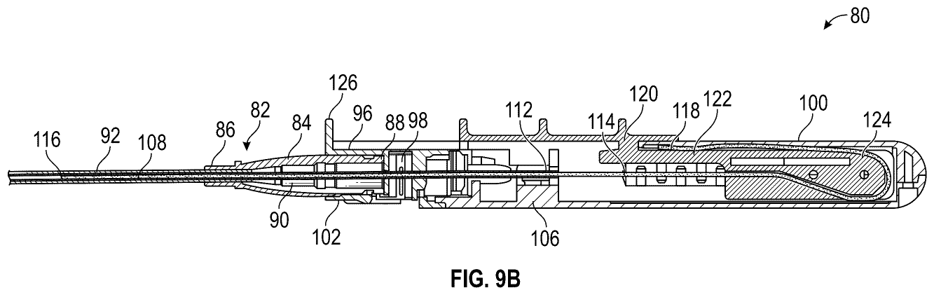

Referring now to FIGS. 9A-9B, a catheter system 80 is illustrated, according to some embodiments. In some embodiments, the catheter system 80 may be used for arterial or venous access. In some embodiments, the catheter system 80 may be similar or identical to the catheter system 10 of FIGS. 1-8 in terms of one or more components and/or operation.

In some embodiments, the catheter system 80 may include a catheter assembly 82, which may include a catheter hub 84. In some embodiments, the catheter hub 84 may include a distal end 86, a proximal end 88, and a lumen 90 extending through the distal end 86 of the catheter hub 84 and the proximal end 88 of the catheter hub 84. In some embodiments, the catheter assembly 82 may include a catheter 92, which may include a distal tip 94 and may extend from the distal end 86 of the catheter hub 84. In some embodiments, the catheter assembly 82 may be integrated, having an extension tube integrated with a side port, such as, for example, BD NEXIVA™ Closed IV Catheter System, available from Becton Dickinson and Company of Franklin Lakes, New Jersey. In other embodiments, the catheter assembly 82 may be non-integrated, such as, for example, the BD INSYTE™ AUTOGUARD™ Shielded IV Catheter, available from Becton Dickinson and Company of Franklin Lakes, New Jersey.

In some embodiments, the catheter system 80 may include a safety housing 96 disposed proximal to and/or coupled to the catheter hub 84. In some embodiments, the catheter system 80 may include a needle safety clip 98 disposed within the safety housing 96. In some embodiments, the catheter system 80 may include a main housing 100, which may include a distal end 102 and a proximal end 104.

In some embodiments, the catheter system 80 may include a needle hub 106 fixed within the main housing 100. In some embodiments, the catheter system 80 may include an introducer needle 108, which may include a sharp distal tip 110 and a proximal end 112. In some embodiments, the proximal end 104 of the introducer needle 108 may be secured within the needle hub 106.

In some embodiments, the catheter system 80 may include a guidewire 114, which may include a first end 116 and a second end 118. In some embodiments, the first end 116 of the guidewire 114 may be configured to advance distally through the introducer needle 108. In some embodiments, the second end 118 of the guidewire 114 may be fixed within the main housing 100.

In some embodiments, the catheter system 80 may include a guidewire slider 120. In some embodiments, the catheter system 80 may include a guidewire carriage 122, which may be permanently coupled to or monolithically formed as a single unit with the guidewire slider 120.

In some embodiments, the guidewire slider 120 may be configured to move from a proximal position, illustrated, for example, in FIGS. 9A-9B, to a distal position to distally advance the guidewire 114. In some embodiments, when the guidewire slider 120 is disposed in the distal position, the guidewire 114 may be advanced beyond the distal tip 94 of the catheter 92. In some embodiments, the safety housing 96 may include a pusher element 126 or tab that extends upwardly away from a longitudinal axis of the catheter system 80.

In some embodiments, the guidewire carriage 122 may include a U-shaped slot 124 therethrough. In some embodiments, the guidewire 114 may be disposed in the U-shaped slot 124 in a double-throw configuration such that there is a 2:1 advancement ratio between the first end 116 of the guidewire 114 and the guidewire slider 120. In some embodiments, in response to the guidewire slider 120 moving from the proximal position to the distal position, the guidewire slider 120 may be configured to contact or engage the safety housing 96, such as the pusher element 126, such that further distal advancement of the guidewire slider 120 to the distal position advances the catheter 92 and the guidewire 114 at a same time. Thus, in some embodiments, the catheter system 80 may use a single slider, the guidewire slider 120, to advance the guidewire 114 and the catheter 92.

In some embodiments, the guidewire slider 120 may travel a first distance between the proximal position and the distal position and then a second distance to advance the guidewire 114 and the catheter 92 at a same time. In some embodiments, the second distance may be about 0.25 inches. In some embodiments, the first distance and the second distance may vary. In some embodiments, after the guidewire 114 and the catheter 92 are advanced together, the catheter 92 may be advanced further into the blood vessel without the guidewire 114 by the clinician grabbing the pusher element 126, the catheter assembly 82, or the safety housing 96.

In some embodiments, the needle safety clip 98 may be similar to the needle safety clip 26 and may include a V-shaped clip with a pawl inserted into a groove of the catheter hub 84. In some embodiments, in response to proximal withdrawal of the introducer needle 108 past the needle safety clip 98, the V-shaped clip may expand without compression by the introducer needle 108, causing release of the pawl from the groove and uncoupling the safety housing 96 from the catheter hub 84.

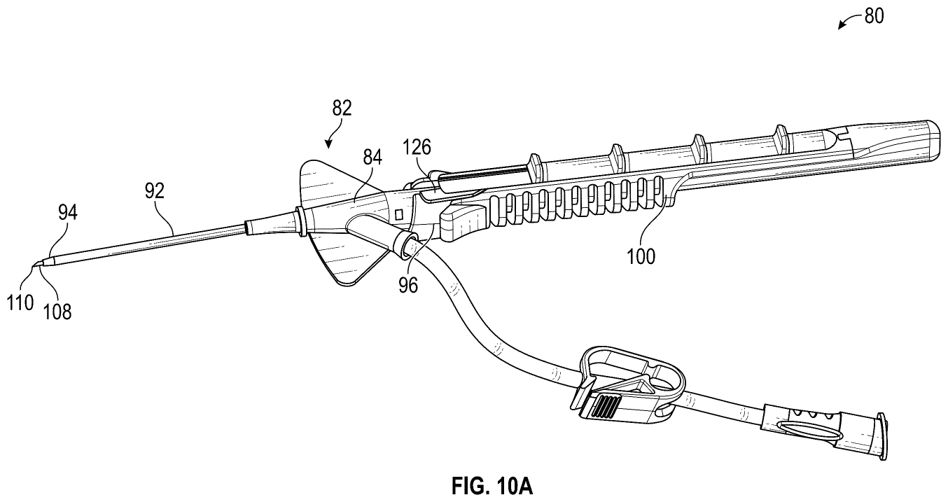

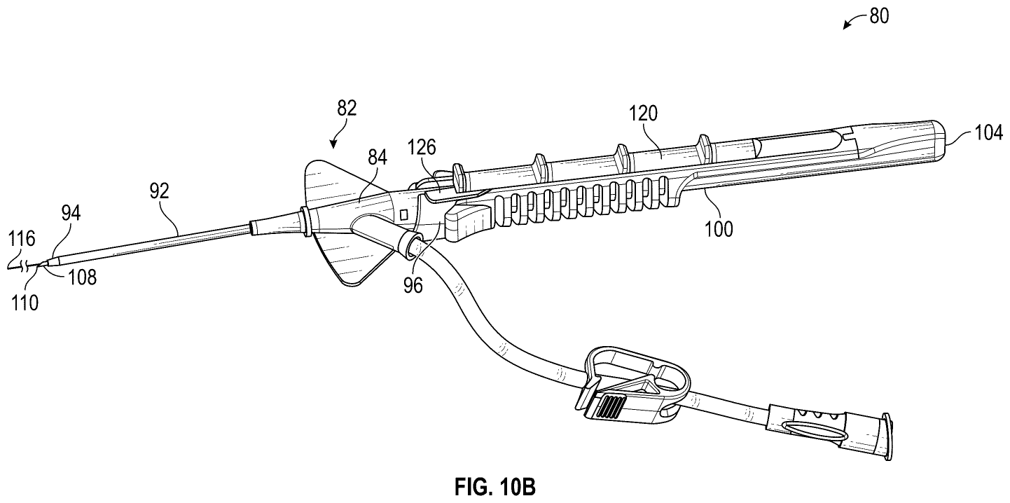

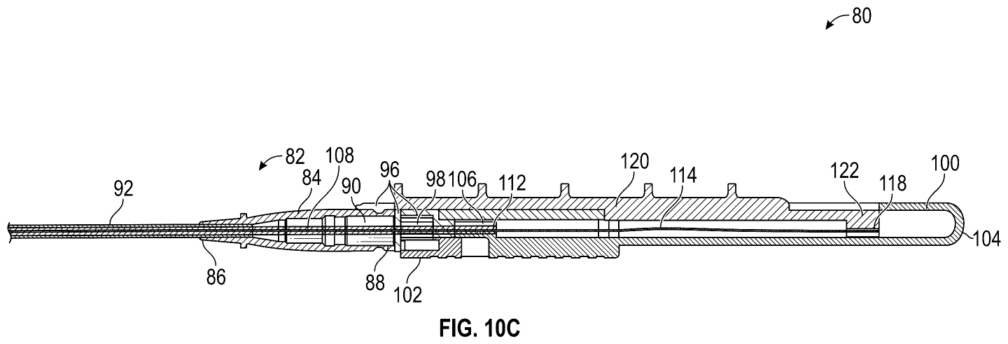

Referring now to FIGS. 10A-10C, the catheter system 80 may include the guidewire 114 that is single throw with respect to the guidewire carriage 122 such that there is a 1:1 advancement ratio between the first end 116 of the guidewire 114 and the guidewire slider 120. In some embodiments, in response to the guidewire slider 120 moving from the proximal position to the distal position, the guidewire slider 120 may be configured to contact or engage the safety housing 96, such as the pusher element 126, such that further distal advancement of the guidewire slider 120 to the distal position advances the catheter 92 and the guidewire 114 at a same time. Thus, in some embodiments, the catheter system 80 may use a single slider, the guidewire slider 120, to advance the guidewire 114 and the catheter 92.

In some embodiments, the guidewire slider 120 may travel a first distance between the proximal position and the distal position and then a second distance to advance the guidewire 114 and the catheter 92 at a same time. In some embodiments, the first distance may be about one inch, and the second distance may be about one inch. Thus, in some embodiments, a single slider, the guidewire slider 120, may advance the guidewire 114 a total of about two inches, and the catheter 92 a total of about one inch. In some embodiments, the first distance and the second distance may vary. In some embodiments, after the guidewire 114 and the catheter 92 are advanced together the second distance, the catheter 92 may be advanced further into the blood vessel without the guidewire 114 by the clinician grabbing the pusher element 126, the catheter assembly 82, or the safety housing 96.

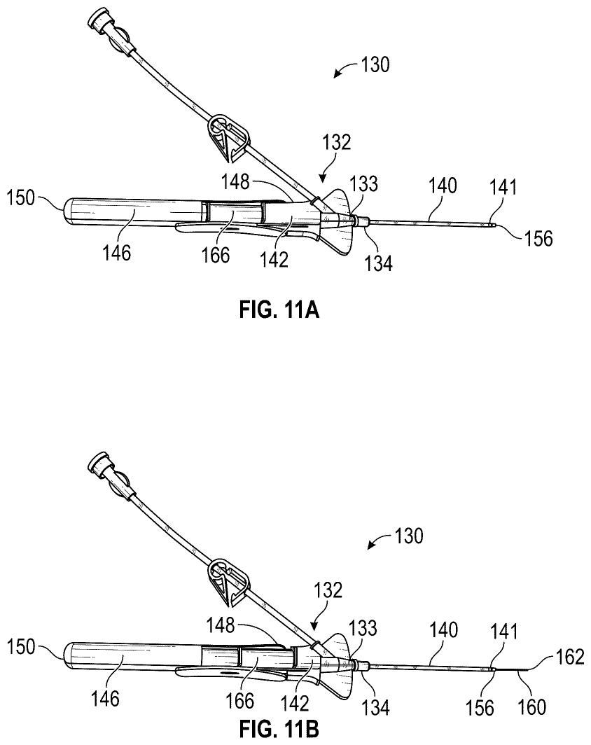

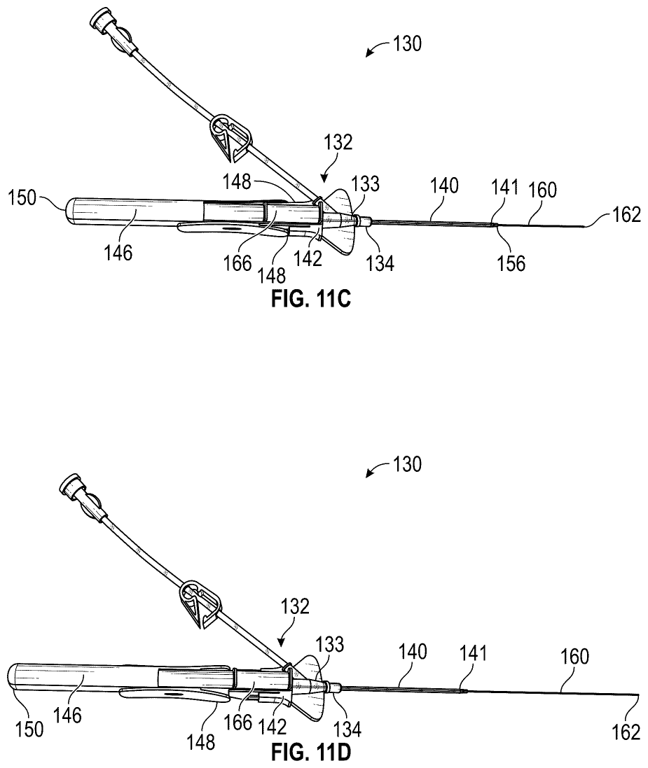

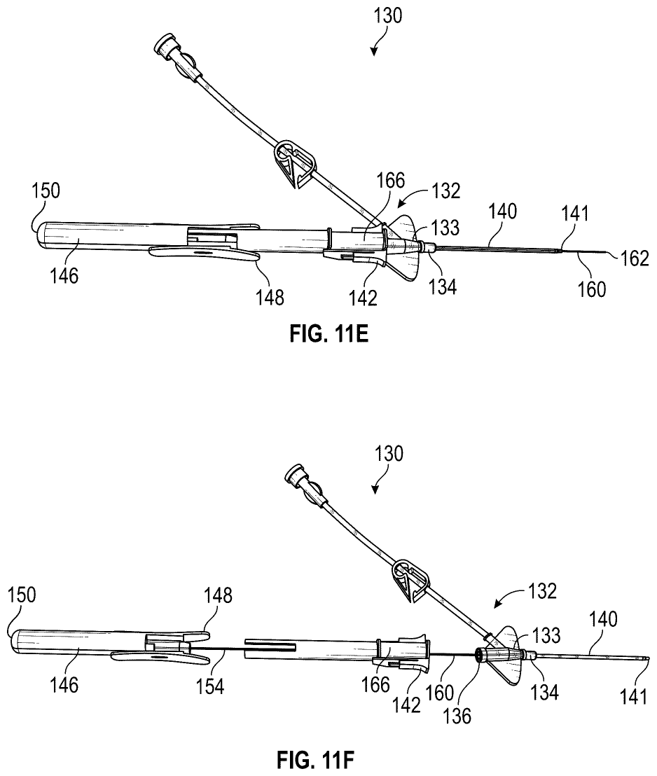

Referring now to FIGS. 11-13, in some embodiments, a catheter system 130 may include a catheter assembly 132, which may include a catheter hub 133. In some embodiments, the catheter system 130 may be used for arterial or venous access. In some embodiments, the catheter system 130 may be similar or identical to the catheter system 10 of FIGS. 1-8 and/or the catheter system 80 of FIGS. 9-10 in terms of one or more components and/or operation.

In some embodiments, the catheter system 130 may be integrated, having an extension tube integrated with a side port, such as, for example, BD NEXIVA™ Closed IV Catheter System, available from Becton Dickinson and Company of Franklin Lakes, New Jersey. In other embodiments, the catheter system 130 may be non-integrated, such as, for example, the BD INSYTE™ AUTOGUARD™ Shielded IV Catheter, available from Becton Dickinson and Company of Franklin Lakes, New Jersey.

In some embodiments, the catheter hub 131 may include a distal end 134, a proximal end 136, and a lumen 138 extending through the distal end 134 of the catheter hub 131 and the proximal end 136 of the catheter hub 131. In some embodiments, the catheter assembly 132 may include a catheter 140, which may include a distal tip 141 and may extend from the distal end 134 of the catheter hub 131. In some embodiments, the catheter assembly 132 may include a safety housing 142 disposed proximal to the catheter hub 131. In some embodiments, the catheter assembly 132 may include a needle safety clip 144 disposed within the safety housing 142.

In some embodiments, the catheter system 130 may include a main housing 146, which may include a distal end 148 and a proximal end 150. In some embodiments, the catheter system 130 may include a needle hub 152 fixed within the main housing 146. In some embodiments, the catheter system 130 may include an introducer needle 154, which may include a sharp distal tip 156 and a proximal end 158. In some embodiments, the proximal end 158 of the introducer needle 154 may be secured within the needle hub 152.

In some embodiments, the catheter system 130 may include a guidewire 160, which may include a first end 162 and a second end 164. In some embodiments, the first end 162 of the guidewire 160 may be configured to advance distally through the introducer needle 154. In some embodiments, the second end 164 of the guidewire 160 may be fixed within the main housing 146. In some embodiments, the catheter system 130 may include a guidewire slider 166. In some embodiments, the catheter system 130 may include a guidewire carriage 168. In some embodiments, the guidewire carriage 168 may include a U-shaped slot 170 therethrough.

In some embodiments, the guidewire slider 166 may be configured to move from a proximal position to a distal position to distally advance the guidewire 160. In some embodiments, when the guidewire slider 166 is disposed in the distal position, the guidewire 160 may be advanced beyond the distal tip 141 of the catheter 140. In some embodiments, in response to the movement of the guidewire slider 166 from the proximal position to the distal position, the guidewire carriage 168 may uncouple from the guidewire slider 166 such that further advancement of the guidewire slider 166 with respect to the main housing 146 does not advance the guidewire 160. In some embodiments, the distal position may correspond to when the guidewire 160 has reached its maximum travel with respect to the catheter 140. In some embodiments, when the guidewire slider 166 is in the proximal position, the guidewire 160 may be disposed in the U-shaped slot 170 in a double-throw configuration such that there is a 2:1 advancement ratio between the first end 162 of the guidewire 160 and the guidewire slider 166. For example, one inch of movement of the guidewire slider 166 equals two inches of movement of the first end 162 of the guidewire 160.

In some embodiments, the guidewire carriage 168 may include a tab 172 that interacts with a feature 174 on an inner surface 176 of the guidewire slider 166 such that the guidewire slider 166 pushes the guidewire carriage 168 when the guidewire slider 166 moves distally from the proximal position. In some embodiments, in response to the movement of the guidewire slider 166 from the proximal position to the distal position, the tab 172 may be aligned with a recess 178 in the main housing 146 such that the tab 172 lowers into the recess 178 and the guidewire carriage 168 uncouples from the guidewire slider 166. In some embodiments, the recess 178 may be disposed in a bottom portion 180 of the main housing 146. In some embodiments, the guidewire carriage 168 may be disconnected from the guidewire slider 166 when the guidewire 160 reaches maximum travel with respect to the catheter 140, thereby allowing the guidewire slider 166 to continue to travel with the catheter 140. In some embodiments, the second end 164 of the guidewire 160 may be fixed or secured within a pocket 182 within an upper portion 184 of the main housing 146.

In some embodiments, when the guidewire slider 166 is in the distal position, the guidewire slider 166 may be configured to contact or engage the safety housing 142 such that further distal advancement of the guidewire slider 166 from the distal position advances the catheter 140 with respect to the main housing 146. In some embodiments, the guidewire slider 166 may be configured to contact or engage the safety housing 142 between the distal position and the proximal position to advance the catheter 140 and the guidewire 160 at a same time. In some embodiments, in response to proximal withdrawal of the introducer needle 154 and activation of the needle safety clip 144, the catheter hub 131 may be configured to advance distally with respect to the guidewire slider 166 and the safety housing 142.

In some embodiments, the catheter system 130 may facilitate actuation of the guidewire 160 and the catheter 140 with a single slider, the guidewire slider 166. Moreover, in some embodiments, the guidewire slider 166 may facilitate the clinician using one hand to advance both the guidewire 160 and the catheter 140 without having to change grip. In some embodiments, the clinician can hold an ultrasound device with one hand and insert the catheter system 130 with another hand. In some embodiments, after confirming placement of the catheter 140 within a blood vessel, the catheter system 130 can be held firmly against skin of the patient while the clinician uses a finger to advance the guidewire slider 166 and guidewire 160. In some embodiments, after the guidewire 160 is advanced, the clinician may continue to push on the guidewire slider 166 without changing grip to thread the catheter 140 into the blood vessel, such as a vein or artery. In some embodiments, while the clinician holds the catheter system 130 firmly in place against the skin, the clinician may use a finger to remove the introducer needle 154 that is shielded. In some embodiments, the clinician may open a connector disposed at an end of an extension tube extending from a side port of the catheter hub 131 and remove air from the catheter system 130 before making connection to pressure transducer tubing at the connector. In some embodiments, when the catheter 140 is inserted into an artery, pulsatile flow may be observed in the extension tube and/or the connector, confirming to the clinician placement within the artery.

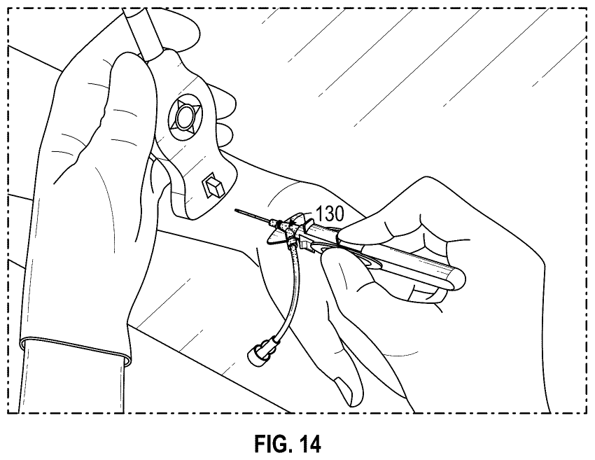

Referring now to FIG. 14, a clinician is illustrating using a single-handed technique to access the blood vessel, visualize blood vessel confirmation, advance a guidewire, and advance a catheter into the blood vessel. At a same time, the clinician may use a second hand to hold an ultrasound probe and continuously maintain blood vessel confirmation. In response to the catheter being inserted into the blood vessel, blood may flow through a notch or an external groove in the introducer needle and within one or more of the catheter, a lumen of a catheter hub, and a side port of the catheter hub, where the blood may be visualized by the clinician to confirm the insertion into the blood vessel. The catheter system 130 of FIG. 14 may be replaced with the catheter system 10 of FIGS. 1-8 or the catheter system 80 of FIGS. 9-10, according to some embodiments.

All examples and conditional language recited herein are intended for pedagogical objects to aid the reader in understanding the invention and the concepts contributed by the inventor to furthering the art and are to be construed as being without limitation to such specifically recited examples and conditions. Although embodiments of the present inventions have been described in detail, it should be understood that the various changes, substitutions, and alterations could be made hereto without departing from the spirit and scope of the invention.

What is claimed:

1. A catheter system, comprising:
a catheter assembly, comprising:
a catheter hub, comprising a distal end, a proximal end, and a lumen extending through the distal end of the catheter hub and the proximal end of the catheter hub; and
a catheter comprising a distal tip and extending from the distal end of the catheter hub; and
a needle safety clip disposed within a safety can disposed proximal to the catheter hub;
a housing, comprising a distal end, a proximal end, and a slot disposed between the distal end of the housing and the proximal end of the housing;
a needle hub fixed to the housing;
an introducer needle comprising a sharp distal tip and a proximal end, wherein the proximal end of the introducer needle is secured within the needle hub;
a catheter hub slider comprising a flexible arm within a distally-extending arm;
a guidewire comprising a first end and a second end, wherein the first end is configured to advance distally through the introducer needle;
a guidewire slider;
wherein the catheter system is configured to move from a first position to a second position, wherein when the catheter system is in the first position:
the guidewire slider is disposed in a guidewire slider proximal position and configured to slide from the guidewire slider proximal position to a distal position to distally advance the guidewire, wherein the guidewire slider comprises a tab extending through the slot and configured to move the guidewire slider between the guidewire proximal position and the distal position when the catheter system is in the first position;
the catheter hub slider is disposed in a catheter hub slider proximal position;
the flexible arm of the catheter hub slider contacts the needle hub to prevent the catheter hub slider from sliding from a catheter hub slider proximal position to a distally-advanced position;
wherein when the catheter system is in the second position:
the guidewire slider is disposed in the distal position such that the guidewire is advanced beyond the distal tip of the catheter;
the catheter hub slider is disposed in the catheter hub slider proximal position;
the distally-extending arm of the catheter hub slider is coupled to the catheter assembly to prevent distal movement and proximal movement of the catheter hub slider with respect to the catheter hub;
wherein in response to the movement of the catheter system from the first position to the second position, the guidewire slider pushes out on the flexible arm such that the flexible arm is removed from contact with the needle hub, and the catheter hub slider is configured to slide from a catheter hub slider proximal position to a distally-advanced position, wherein the catheter hub slider comprises a tab extending through the slot and configured to move the catheter hub slider from the catheter hub slider proximal position to the distally-advanced position in response to movement of the catheter system from the first position to the second position.

2. The catheter system of claim 1, wherein the second end of the guidewire is fixed within the housing, wherein the guidewire slider comprises a U-shaped slot therethrough, wherein when the catheter system is in the first position, the guidewire is disposed in the U-shaped slot in a double-throw configuration such that there is a 2:1 advancement ratio between the first end of the guidewire and the guidewire slider.

3. The catheter system of claim 1, wherein the catheter is configured to move from the second position to a third position by moving the tab of the catheter hub slider distally towards the tab of the guidewire slider but not contacting the tab of the guidewire slider, wherein when the catheter system is in the third position:
the guidewire slider is disposed in the distal position such that the guidewire is advanced beyond the distal tip of the catheter;
the catheter hub slider is disposed in the distally-advanced position, wherein distal advancement of the catheter hub slider advances the catheter assembly.

4. The catheter system of claim 3, wherein the distally-advanced position of the catheter hub slider is a partially distally-advanced position, wherein the safety can comprises a groove, wherein the housing further comprises an inner ramp, wherein the catheter system is configured to move from the third position to a fourth position by moving the tab of the catheter hub slider distally towards the tab of the guidewire slider to contact the tab of the guidewire slider, wherein when the catheter system is in the fourth position:
the guidewire slider is disposed in the distal position such that the guidewire is advanced beyond the distal tip of the catheter;
the catheter hub slider is disposed in a fully distally-advanced position;
wherein in response to movement of the catheter system from the third position to the fourth position, the distally-extending arm of the catheter hub slider contacts the inner ramp on an inner surface of the housing to remove the distally-extending arm from the groove of the safety can such that the catheter hub slider is uncoupled from the catheter assembly.

5. The catheter system of claim 4, wherein the distally-extending arm comprises a hook and an outwardly-extending protrusion, wherein in response to movement of the catheter system from the third position to the fourth position, the outwardly-extending protrusion of the catheter hub slider contacts the inner ramp on the inner surface of the housing to remove the hook from the groove of the safety can such that the catheter hub slider is uncoupled from the catheter assembly.

6. The catheter system of claim 5, wherein the distally-extending arm is a first distally-extending arm, wherein the catheter hub slider further comprises a second distally-extending arm opposite the first distally-extending arm, wherein in response to movement of the catheter system from the third position to the fourth position, the second distally-extending arm of the catheter hub slider contacts the inner ramp on the inner surface of the housing to remove the second distally-extending arm from the groove of the safety can such that the catheter hub slider is uncoupled from the catheter assembly.

7. The catheter system of claim 4, wherein when the catheter system is in the fourth position, the housing may be moved proximally with respect to the catheter assembly to withdraw the introducer needle from the catheter assembly, wherein in response to proximal withdrawal of the introducer needle past the needle safety clip, the needle safety clip uncouples from the catheter hub.

8. The catheter system of claim 4, wherein when the catheter system is in the first position, the second position, the third position, and the fourth position, the introducer needle extends through the catheter.

9. The catheter system of claim 1, wherein the flexible arm comprises a fin tapered inwardly in a distal direction, wherein in response to the movement of the catheter system from the first position to the second position, the guidewire slider pushes out on the fin such that the flexible arm is removed from contact with the needle hub, and the catheter hub slider is configured to slide from the catheter hub slider proximal position to the distally-advanced position.

10. The catheter system of claim 1, wherein the flexible arm is a first flexible arm, wherein the catheter hub slider comprises a second flexible arm opposite the first flexible arm, wherein the second flexible arm of the catheter hub slider contacts the needle hub to prevent the catheter hub slider from sliding from the catheter hub slider proximal position to the distally-advanced position, wherein in response to the movement of the catheter system from the first position to the second position, the guidewire slider pushes out on the second flexible arm such that the flexible arm is removed from contact with the needle hub, and the catheter hub slider is configured to slide from a catheter hub slider proximal position to a distally-advanced position.

11. The catheter system of claim 1, wherein the catheter assembly further comprises another safety clip disposed within the safety can and configured to trap the sharp distal tip of the introducer needle in response to proximal withdrawal of the introducer needle.

12. A catheter system, comprising:
a catheter assembly, comprising:
a catheter hub, comprising a distal end, a proximal end, and a lumen extending through the distal end of the catheter hub and the proximal end of the catheter hub; and
a catheter comprising a distal tip and extending from the distal end of the catheter hub; and
a safety housing disposed proximal to the catheter hub;
a needle safety clip disposed within the safety housing;
a main housing, comprising a distal end and a proximal end;
a needle hub fixed within the main housing;
an introducer needle comprising a sharp distal tip and a proximal end, wherein the proximal end of the introducer needle is secured within the needle hub;
a guidewire comprising a first end and a second end, wherein the first end is configured to advance distally through the introducer needle, wherein the second end of the guidewire is fixed within the main housing;
a guidewire slider; and
a guidewire carriage, wherein the guidewire carriage comprises a U-shaped slot therethrough;
wherein the guidewire slider is configured to move from a proximal position to a distal position to distally advance the guidewire, wherein when the guidewire slider is disposed in the distal position, the guidewire is advanced beyond the distal tip of the catheter, wherein in response to the movement of the guidewire slider from the proximal position to the distal position, the guidewire carriage uncouples from the guidewire slider such that further advancement of the guidewire slider with respect to the main housing does not advance the guidewire, wherein when the guidewire slider is in the proximal position, the guidewire is disposed in the U-shaped slot in a double-throw configuration such that there is a 2:1 advancement ratio between the first end of the guidewire and the guidewire slider.

13. The catheter system of claim 12, wherein the guidewire carriage comprises a tab that interacts with a feature on an inner surface of the guidewire slider such that the guidewire slider pushes the guidewire carriage when the guidewire slider moves distally from the proximal position.

14. The catheter system of claim 13, wherein in response to the movement of the guidewire slider from the proximal position to the distal position, the tab is aligned with a recess in the housing such that the tab lowers into the recess and the guidewire carriage uncouples from the guidewire slider.

15. The catheter system of claim 14, wherein when the guidewire slider is in the distal position, the guidewire slider is configured to engage the safety housing such that further distal advancement of the guidewire slider from the distal position advances the catheter with respect to the main housing.

16. The catheter system of claim 12, wherein the guidewire slider is configured to engage the safety housing between the distal position and the proximal position to advance the catheter and the guidewire at a same time.

17. The catheter system of claim 12, wherein in response to proximal withdrawal of the introducer needle and activation of the needle safety clip, the catheter hub is configured to advance distally with respect to the guidewire slider and the safety housing.

18. A catheter system, comprising:
a catheter assembly, comprising:
a catheter hub, comprising a distal end, a proximal end, and a lumen extending through the distal end of the catheter hub and the proximal end of the catheter hub; and
a catheter comprising a distal tip and extending from the distal end of the catheter hub; and
a safety housing disposed proximal to the catheter hub;
a needle safety clip disposed within the safety housing;
a main housing, comprising a distal end and a proximal end;
a needle hub fixed within the main housing;
an introducer needle comprising a sharp distal tip and a proximal end, wherein the proximal end of the introducer needle is secured within the needle hub;
a guidewire comprising a first end and a second end, wherein the first end is configured to advance distally through the introducer needle, wherein the second end of the guidewire is fixed within the main housing;
a guidewire slider; and
a guidewire carriage permanently coupled to the guidewire slider;
wherein the guidewire slider is configured to move from a proximal position to a distal position to distally advance the guidewire, wherein when the guidewire slider is disposed in the distal position, the guidewire is advanced beyond the distal tip of the catheter, wherein when the guidewire slider moves from the proximal position to the distal position, the guidewire slider is configured to engage the safety housing such that further distal advancement of the guidewire slider to the distal position advances the catheter and the guidewire at a same time.

19. The catheter system of claim 18, wherein the guidewire carriage comprises a U-shaped slot therethrough, wherein the guidewire is disposed in the U-shaped slot in a double-throw configuration such that there is a 2:1 advancement ratio between the first end of the guidewire and the guidewire slider.

20. The catheter system of claim 18, wherein the guidewire is single throw with respect to the guidewire carriage such that there is a 1:1 advancement ratio between the first end of the guidewire and the guidewire slider.
